SEQUENCE LISTING (10 sequences)

SEQ ID NO: 1 - DNA, 1450 bp, Gossypium hirsutum
  source 1..1450: mol_type = genomic DNA
tgtcgtgtgggtatagaaccactaccttagaagatgttgcattgcaactcaggctctcaattgacggtggttatgcaatcacagattcaagtataactcgggctccagttgacaatggttatgtagtcacgggttcaagtttcatgccatcggaagagaatgttttataaacctcatacataagtttgagatcataatcatcggggaaaaatagggctttccagtataatcaatttattatttttctccatctccattaaagtagacatcaccgtttgaaacaggagtcgaggcggcagtgattgctgtgattaaggaactcgattaagaattacaatcgttgctgctccaactcgagtttcagctttttctccaccgtcacaatcattcctcccccctgaaagtcttcttgcacccaacactgtcacaggttatggacaatgaggttaaagatactacctttggtggagatgaagaaaaataataagttgattatactgaaaaacccttttggttttccctatgatggtctcaaacttatgtatgagatttataaagtatcctcttttgatatcatgcaatttgaacccatcaccacataactaccgccaacttaggcctaagttatacatcaacttgtgatcgtttaaccaacgtcaactgagagctctagctatacttcaacttgtgactgtgtagacaccatgaactaggagcctgagttgaaatgtgatgagcttctaaggtgatggtttcatctgcacacggtagcatgaaatgtgtgagtctttgagcgtcattgcatcaccgtgaactcaaacataccttaaaattatcataatcggtatctctttcacaaacacaaataaatttttctcctcaaatttccaacactcaacacaaaattttttttttttagaaacaaaaaacgtttcctacacgtattttctgtttctttctcccaaatgcacctattttcttaaatgacaaaaaaaaatctaaattcataattaacttttaaattaatctttttttatctaattaacccgaaagttaagtgatatttattctaaaaatagtaaaacgtatttcttaatgtcaaggctctgcgtggatgcggtgaacaatccatagccgacagcagctaccggcttcgtgtcgccaccttatcttatcttgtcttggatttacctgtctaattgattaaaatacttaaacaaacatcctaagccttacatgtttgccatatggctgaaacgtgtaagcaattcaatgatccaaggagcatacgtgtcagatttacttgtatacgtggcagctgagcaactatcatttgggtcctattaatggcaagaattttggagttcgagcatcaacttcatcagcaagcaaatataccctgaaaaccctttcactaaatattgaagatgcagtcaatgaaggatgctgc

SEQ ID NO: 2 - DNA, 1679 bp, Gossypium barbadense
  source 1..1679: mol_type = genomic DNA
catataaaagtttaggggattggagaggttgccacccaatgtcacggcattcccatcagtctagctgaaagtgtgatatttatagatcatttatcaaatgaagttatcagtgaaatgccaactttagtgcttatcaaagtgataataagcactgagccaatgcatgttgacattagtctttgaaatctaaaccagaaattatctcacataatgtttacattgtaaaatcacaggctaactatcactcttaagaatccaacaaggttttcattagggaaatgatagccaagatgaaccacaccatattaagagcaggttccatattgggaagttttaggactaatgtatcatctatgaggttccaaatgagttttaggattagtgtatgaaacgaaattttatggctacaatacatttccaagtactgtttccttttaaacatcccaataaattggaaggacatatatctttgtctacggttcagaccacagagctatgggttattcttgtattaaaattgttcgaattcatcttataattatattcaatacatataattagtgagtcattttagatcatttctatattcatatttgttgagggagattgatttctttggaagaaattttcaaagtgtcatccttaaggttgccgatttggtccctcaacaatggaaggtattatcctcttagggagggttttgtcgcccctccccgtcctcaaggatgccgacccgatccctcaacaatggaaggtattgtccctcaacagatgacctatccgtaggctaggggttcgaatcctaccaagggtgggatgctcacgtcacctcttggtcagcagtcgtaaatccacgtacggttaaggtgtccccttccgtgaattggacacggctctttgtggaggagggacaaaatcctcccctgaggactataccttccgttgttgagagactgaatcggcatccccaagaatgacacttcaaaaatttctcctaaaagaactgatctccctcaataatattgtgtttgtattttgttttcttgtgttgtgttatatttttaatctttttttcaaatcatatttgtgcgttttatccaaaattaataaattatcaaatcttgataagagggatgtcttgggagacagatactaatgtttaagcaaacattaatctcatgcctataccaacctattgattttatatttatatacgtctcctgtggtaagtgaagatggtcccaattcatttatctctcttcattggattacatctccacttggacttacccattaatcagtaatttcttttctccctaaaagttgataattatcgtccaatatcctagtcttgtgaacaaaattatctgatgatgataaccaaatataatatgcctgattgtaatccaaagtgacccgcccatgttccatgagttgaagtcctcgttagaaagaccatttcaagttcagcattgtttcagcaattcaaaagttaataattaatacaaacatataaatgcacctgctgtcatcaactaacctcatgcaggttcacctgtttcatttacttcacctagttagtgctttatattgatcatccccctcattcttctcaactcacacatcttccaatctttcattctttgctgttgaagtagtaatatctatagcatacagag

SEQ ID NO: 3 - DNA, 1351 bp, Gossypium barbadense
  source 1..1351: mol_type = genomic DNA
atctacctcttattgaatttgcatacaataatagctttcaattgagtatcaaaatggcaccttacgaagctttgtacagtcgtaaatgtcatacacctttgtactagactgagctcagtgaaaataagatacacgtggtcgatttgattagagaaactgaacagaaagtgaaagtgatccatgaaagtttgaaaacaacattagatcgtcagaaatcgtatgcagatttgaaatgtaaagacatagagtttcagatcggggatagagtgtttttgaaagtctcaccgtggaggaaaataatcagattcggtcgtaaaggcaaattgagtccgagatttgttggaccatacaagattatagagaggattggaccggttgcgtatagactgctattaccacctgagctggaaaagatccataatgtacttcatgtttcgatgctttgacgatatagatccgaccccttgcatgtcattagtccatcagagattgagattcattcaaatttgacttacgaggaagaactgattcatattttggctcatgcaattaaagagttacaaaataataaaattttgttggttaaagtactgtgggataaacatggtgttgaggaagcaacgtgcgagccagaggatacaatgaaagagtgttatccgaacctattcaccggtaagatttttggggacgaaaatccctaaggggggagagttgtagcagaccgattagaccctaatcggaacggtgattttgggaccacaaatccgagtcagaaaaatatttttccgtgtttattatatgtgaatttgcatgtgtgaacgtttcctacgaaaatttaattgtttgtgtgcgtaatttgataaaaaggacttaatcgcgtaaattgaaaatttgataatttaaatcataaaggctaaatttttagtggctttttaatatgaggtgtttatgttgcaaattgcccattaatcagtaatttcttttctccctaaaagttgataattatcgtccaatatcctagtcttgtgaacaaaattatctgatgatgataatcaaatataatatgcctgattgtaatccaaagtgacccgcccatgttccatgagtcgaagtcctcgttagaaagaccatttcaagttcagcattgtttcagcaattcaaaagttaataattaatacaaacatataaatgcacctgctgtcatcaactaacctcatgcaggttcacctgtttcatttacttcacctagttagtgctttatattgatcatccccctcattcttctcaactcacacatcttccaatctttcattctttgctgttgaagtagtaatatctatagcatacagag

SEQ ID NO: 4 - DNA, 2080 bp, Gossypium hirsutum
  source 1..2080: mol_type = genomic DNA
acgtcgcatgctcccggccgccatggcggccgcgggaattcgatttgtgtcaaagacgactacttgagactgtcaaaacctatacaacaaaaaaaaattaaaatcattctcttaatttacccatttgaaatggatgccacacataccatttgaaatatcttaaagagaactttcatttgacaccaccaatgttgtcggaacctcaccagaatttagtaactatactaacccaaatgaaatacatgactttcacgaaaaatagattaaaacacaagaaatctaaaccaaagttatccttaatgtctaaaatccactagttatttgcccttcctttttctttcttcctcttctctatggttagcttctaccaaattgtcaagatcatcatctaacataaaaataccgtaaatgattcttttctctttttttctagtctattctttttattagaaaaaactataacctacataataaagttgagcttgaaaagaaaattggctaatggaccacatatttaaataatacccatttaaaaatcatgaatattacaaatgtaataacacctgttaaaggaaaggaggtcacaaacaaaagtgatttgtacagagttttttatcaatgcaaagttggcgatatgatatagcattgaataaaaccaataaccatttctttctctctctatatatatacacgtctttaggatcttttatgatgtacaaatttttcataattttaatttttaaattatcgtggaggtgttacatgaggagaagatccctataacactcaagccaaagccagatgagtcatgaaatgatgtttagtatgttgcataatcctaaatacctagtaaatatacaaggaaatgtcattatttgtttactattttactggaaaaatattcctgttgtatctatgtggctatgccatcaacttgttatcatctttcatacaatgttttcacaatgtgaagcacaaggctatcaacctttgtaaaaaattgtatctatcttgccttttcttcttaactgattggggtataagtgcaaattctatgttacatggactcgtctatgagtattgaatatgggtaacacgaaataagcaaaccctagtaaataagggacctcctaatggtgtgttgtaagcttaggtggaagtgtagaaaagaggaaagggaaattgattgtcccaaaaaagtaagtgatggatttccgaatttagataaatgtgcatttgctaaggcattacggcccctacgtgcacggatgtgatatggcagttagttgttggtcataaatatctttgcattttctagtttgagttcaagtagtggggtttggatgagtgattgggtgcggtgcagtgtagtgcaatgcgtttagtttactttttatctcacactacagtatcattacaatatataatctcaccgtcactgctatttttacactaaccgcaagaaaatgtaccatctcatacaaactaagccttagacttataaacttttaaaactcgcatctaattcatttaattttaaaaaaattaatagattatatttgaattacaacatttgtaatgtattctcttaatccggaagcgatgaactcttttgtttatatatagaaaaaagaacacaataatttgggttatggattttttttaactttaacatttaccattcattttaatcctggaattaatttagcatgaatctaattaagagagctcacaaaatatgtgaagatgtgtgcaaaagctattaagagtaatttatccaaatctgccatccatcatcatctcatcattacctccatatatacaaccagaatcctacacttacagctaagaaaggaatatatatatatttaaaacagcatttgtgtttcaaaaaaatctaatccatatactgcaaaatccttgtgcatcttcttcagagcaaacggcaacaactccaacaaatgtccatgaaaaaagaaggtgaaattctatacaaaaagggattatgggcaatggaggaagacaagttactcaatcactagtgaattcgcggccgcctgcaggtcgaccatatgggagg

SEQ ID NO: 5 - DNA, 12578 bp, synthetic construct
  source 1..12578: mol_type = other DNA
gcggccgcttgcatgcctgcagtaattaatagtaattatgattaattatgagataagagttgttattaatgcttatgaggaataaagaatgattaatattgtttaattttattccgcgaagcggtgtgttatgtttttgttggagacatcacgtgactctcacgtgatgtctccgcgacaggctggcacggggcttagtattacccccgtgccgggatcagagacatttgactaaatattgacttggaataatagcccttggattagatgacacgtggacgctcaggatctgtgatgctagtgaagcgcttaagctgaacgaatctgacggaagagcggacatacgcacatggattatggcccacatgtctaaagtgtatctctttacagctatatcgatgtgacgtaagatgctttacttcgcttcgaagtaaagtaggaaattgctcgctaagttattcttttctgaaagaaattaatttaattctaaattaaattaaatgagtggctataaatagtgtcgatgctacctcacatcgtattcttcttcgcatcgtctgttctggttttaagcgatacgtagaattcctcgagggtaccatcgatcctagggagctcgttaacgtttagcgggggaaaaaggacagttgatctgttgctgtttgcaattttttaaagggtatgttgtcagatgcatgttgtaatgcttgttcatcaacacattatatgacttgcagttgctgatgatggaaacttaaagcttaatactacttttgtttattcacttacaaataccggttgggttctttgtttatcaggaatgctcattgtatgtagctaaaagctggccgtttatagttttattgccctaaatctggtactttatccaaaaactaaatttggaaacatcaaatacttttttcaagaatgataaactcgtacactctctagggtactcctgaaatttaaatcaaaatccaaaaccgcttaggaaggaacatatgtgataagaactgaaatttcgattaactattacaagatagtcggcccaattcgagaggactagtctccgattacaaggagtaaatatcttaatcttgataaacaaaacacatataaaaaacctaaaaatataggaacataatacataaactaaaagttgtgggaacagttacaaatctgcagtctcactccctaaatttgtgagtcacctttcacctccaagttttcgaatgttctcccaccattcactttccctccacccggattccctccaattaatagctgacacaacccgttttgacccaacattgggttcgtatcaatacatccggcccggaaaatcgacttgtcctcaagtcgaaaggaggggaattattgtgccaagcaaaaagccattcgattggaggttgatggatgatttccttgtgtttgaaagcttcaaaagatccggccaaatcagcttttaatgcctcttgaactgtagccacaacaccactttgaaacctcaaatctgttttaattaagcggccgccaccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatcgtcgaccctttccgacgctcaccgggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgtttaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgatatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgaagatcggcggcaatagcttcttagcgccatcccggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgacgatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaattctagggccgctctagaggatcctatgttgtaattttatatggattaatgagaattattattattctgttcttcgtctgtgttttttaagctagcttcctcgaagcagcgtataactttaatttgaatttggttttggcgcgttagtgaaattgcggctgtaaacgtgtcaagttgtgagtggctgaaataagataatagatatattattattgttttaatttaattccgcgaagcgatatgttaagtgataaatgaaacgaagcgttttgatgacgtcatatgtctccgtgcctacgtcagcacggggcttagtattacccccgtgccgggatcagagacatttgaccaatagttgactagtataatagcccttggattaaatgacacgtggacgctcaggatctgtgatgctagtgaagcgcttaagctgaacgaatctgacggaagagcgttcacacttagatctagttagcgtacttagtacgcgttgtcttgggtctataaatagagtgcttctgaacagattgttcagaatttcatagcgccgataacaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggtcgacggtattgagtttatgtctattgtaattgatagaggttctattaagatagaattatgagatgtaattgtgattaatgaataaagagttgttattattctttgaattactccgcgaagcggtgtgttatgtttttgttggaagctttctagactcgactaga

SEQ ID NO: 6 - DNA, 495 bp, Petunia hybrida
  source 1..495: mol_type = genomic DNA
catcaaactaaattaatactctggaatattagcatgcgtgaatgtcgtcgtttgtcaattgaacttcactaatagtgttaaacaatagatacagtgataaagaatagatacagtgatacaaaatcaggtcatcctgctaatcacactacactaatagtgtaaataattaaactaaagatttaaatagaagagggggcatattagtcaagttgaaagcaagtaacacgctttgcaatttttggtgaaattctggtctgcagtaaagcagcgtacaaggccacgtctggtttatatctggaaaaaggccatttcaatgtcattttcttggccagctctttctttcctttaattttaagtgatagttaaagctagggtaggtatttataaggaaagaaagacattttaagtattgagtttttcctcattcattagtgttgtaattccaagactacaaagtctttggaagtacgtgtgaggtttctgaaaggctttgaa

SEQ ID NO: 7 - DNA, 6254 bp, Nicotiana tabacum
  source 1..6254: mol_type = genomic DNA
gtttgacagcttatcatcgattatattagggatttttacacaaatagccggctatattaattgtttactttttctaaccatatacatagattatacattgattatacatgattatacacatttaatatataaattatgcatataatatacattcgctggttatttttagtttaagttatagggtgggaggctatttggattaattctttattatattaaatatttacgaatttcgtgtttgacggtgtaaatcatatgtattgatacttgttgcttcttttattaattttagtgatttagattccctagaaactaccatatgctgtttttaggttcgtataatatgagaaaagtttatttttagtcgcttccaaatatatatttatatactttctccggtccacaataagtgattttttggttgttttcacacagattaagaaattcacattttaacattaaatagcaatgaaattgatcatattaacctttactatttcttcacataaacatttctaacacatactccaacaccatttactccaagggcactgtagtaaaaaaataattaaatcatttttgaaatctaaaaaactcacttattttggaccataaaaaaagggccaaaaaataacttattgtggaccggagagagtaatacactttttggttagcgaatgcaattaatttagacattgtgttatgttccagttaaccgcttccctgcacttctttcaatctatctctcgatagaaaattgtgatactttgcgacttctatcagaggactttttgttttccatgtaacaatctgtcattttcgatggggagatttgcacaaataggctatttatgtgtcccaatttaaattttaaccccatgtcgatcagaacttagccacgagcaccagaagtttgatggatatgtgactttgtcactatccggtttactaatcaagagctatttttattcaaaattggatatctagctaagtataactggataatttgcattaacagattgaatatagtgccaaacaagaagggacaattgacttgtcactttatgaaagatgattcaaacatgattttttatgtactaatatatacatcctactcgaattaaagcgacataggctcgaagtatgcacatttagcaatgtaaattaaatcagtttttgaatcaagctaaaagcagacttgcataaggtgggtggctggactagaataaacatcttctctagcacagcttcataatgtaatttccataactgaaatcagggtgagacaaaattttggtactttttcctcacactaagtccatgtttgcaacaaattaatacatgaaaccttaatgttaccctcagattagcctgctactccccattttcctcgaaatgctccaacaaaagttagttttgcaagttgttgtgtatgtcttgtgctctatatatgcccttgtggtgcaagtgtaacagtacaacatcatcactcaaatcaaagtttttacttaaagaaattagctaaaatggtagctccaaaatgggttttcatttcttttatgattttgctaagcttagcaatatgctctggccagcctgttacctctgatgcaattaaggctaaggaagctgatcatgacaacctcaaagctcacactctgagtaatatcgacgccaaaggctttggaggaggcggtggatttggcattggtggtggttgggccggtggtggtggaggtggtggagatggtggtggttctgacacccctaactacggttataaccctggctgcagtatccatggttgcactgtccctggctttggtttcctacctaaacctgtctttggtgtcccagtctattcccctggttgtggctatgtgtgtccggccgatattcctactggaggaatgactgaatccaaaatcacaggaatatcacaatccgctagactataccgttgcaagccagggccaaatatgtgtgacagtaaagactgtaatgagcttctcctacactttgttttcccaatgcaagacaaacatgacaataaacaagaacatctaagatatggaggacgccgaggtataggtctcactgtgggaggagttggcggttttggaattggttttggtgcttggggtggtggtggtggcggaggaggtggtggttctgatgcccctggttgtagtaacgatggctgtgaccctggttttggctgtcccccgggctgtggttatgcatgtcctgccaacaatcctagtggaggaataactgaattccatatctcaggattatcacgattcgatggaccttacagatgtaggccagatatgtgtgaaagtgaagattgtaatgaacttcttctacactttgtttctccaatgcaacacaaacatgagaaccgacatgatcatatagtagaaaggagtgatgaggaggaagcgcatcatcagtcaaagcagcataaagacgaagacatcataaactaggctctcccacaaaccaaaaaaaaaggaactatatatgtagcttcagccaaaaaactgtatacactgtctaagaatactcacttccaacgaacttaaataaaactagtttacagtggattgggatataatcagttggacaatttgctaaacctcctcatgcactgtaaaaatagacttgctactagtatttggaatataatgctgaatatatttgttgttactttgcctaatgtcaatcagcattcagcaatttctctgtagttagaaaatgaaaggaagaatcaggaaactcatatttaaaggatgaaataatttaaagatcgcgaagcagtcacaatttaatagtaccaggaaaataatctataggaatcacagaactttttgatttatcaaattaaggaagcaaactgggaaaatgtgaaatgaatgaacataatgctgaaagctattgatcagatgattggattgatttcgtaggagcaacatatgatttaagattatttcaacaagatggccataaagtagcatatcatttgtaatttaacattattacactcaaactcaggaagattgtcaatttaccctcaaaacaaagttttaagccttcagtctccttcaaccacagtggcacctgcccaattggcagcacttccccggcgtggaatgctgtggagtttggtgtacaaatccacctggaaaatcacagcattgatgtttccttcatcatcttcgcgtgcaattgcttttactttgtgcagtggatgatcaaagcttattagtccagtgtcacttatgaagatacaccctgttcaatcagcttttatacattaaattttgcttgatatccaaaaaagattgaatcaaacttgaggatttgattaagaaacctggaggaaatggggtatagaattttccacaagaagcttgaatgaactctgaatcatcggaacatactaaagacccatctgcagcagttccccagtgtaacggcacacatccatcgcgatcctacattgcttagagcaacattaagtttatcctaaaaaggatttaagttatatacattgacaatataaagatttttttagtgtagtttaacctagtttaacatgtgatagaaggttagttaccacttttacccggtttccaactaatgtttatcatgtaattaccttataagtgacataactggtaaagttgctaccatgtgatcaggacatcacgggttcgaaccatgaaaacaacctcttacagaaatgcatggtaagactgcgtataatagactcttgatgtccagctcttcccggaccccgcgcatagcgggagcttagtgcaccgggctgcccttaatccttataaatgacatgattgtataaacattttttatgtcgtcagggcaccaaacttaaactcttctaaaaatactatgttgatcatggtgtatagttaagtttaaagaaacagaccaagttatggatcatggaaatggttaatatgaatgttcaataagctaagaaagagatcaattatgtgagaaaaagcattgtgactcaccctggcaagaaaaagagtagaagcttttgagtcaaacagaatgaaagcaaatttgccttcaagttctttgatgacttgatcaggagggtatggcgcccgatcccttaaaactttgtaagcttcaaccataatcatagcctctgtggcttgtcttgaaagaccataatgttttctcaaatcaaaagtgttatctaaagccccagagaatatgcagaagacatcatccatcacaacaatagacctaatgtaaacaaaacaaaacaaaacaagctcagacaacttcatttatagtcaccttgctattgcttcaatcttgaaatactacactagtttttaagaaaagttcacccttttagtatactaatcaatgtgcccaaaaagaaaaaaacaaaacatgttactattactagtactaaacatcaataccttggttgtaaagggttttcatttccatgagaaaaagccatgaaattcccattgaaaaggtggtaaaaagttgagtcttgcttccatgttttgaaactttctgcaatctcttcccttgttttagcctcctttttctgtatacctgcttgaggaaggctcaactctggaggtggcctaccaatggattgctcaaacacagccaacatgtttggacaaagtaaaagctagaaaaatttcaagaatcccaagctaatttcttttttcaagaaccataaaaatcccaggaaagcacattctatgtacttataatatagaagacttgcagctgacatgcttatccaaaagactcttgtaactgaaaacaaagtactgacgtggcatcaacaaagtgatagtgcatctgtcacactctcctttgttcaagatggtactactttaatattttatgggcgaataattactttgggaatttgtaaacaaaatttctttatttctttcattgatgggatagatgtcttggaacatggttttgaagtaccagaaaatatgatatactagtagttttagaaaggtgcatacatgcagtgggttaacaattgaatgtataacttttaatgcggagtaaaagtttattaaaattataaaaataatgaatataaactcataactttaggttcaacgctaaaaaaatttaaaagttgaacccataaaatttaaatcttgaatttgtgtctggttttaaattatgaataggacagtagaaacaagccaagaaagttttgtaaggctgaagctagcagtatatgactttacgcacttcgagaagcaggcaatactggactcttctaaataattgatccacttaaacccaaaattatttgcctatgcaacgaagtctcagaaagcacgtaataatgctatttcaagctttctgttctttcaatgatgaaactaattaagctttttttcagtggaggtcgacgtcaaggtatctttcatgtatgcattattttgatgtaatttgactaataggaagagataataagataatgtaattcccgattgagacattatttgaaaacagcaaaatctgtgggacataaccattagcggaaatgagaaaagaatggttttttgtttgaaagttacccccgcaaacgagattagcttgtgacccgtttggccatagattttgtcaaaataaatttggattttatttggcaaacatatgtttggccatagattttacctacatttcggtaaaatcaagagctggttttgagccaaaatatcactattacattttttaaaaattgcccaaacttttgtattttataaaagagcccactatttattattttgtaacaatgttacttcgtcttctcgatcacgtgatagtgtatcatgcagttcattataaaaatgataattttgtatcaaatttattcatgttcaatactatggtttgcgataatataatgaatgttattgataatggtaatgttgggtatttgtaatagtttttaaaacttgtgggtataaatcatatttcatattttttcaaaataaatttgaaaaatatgttttgaaaactgatgtccaaacacattttcatcttcaaaccaaacttcgcccaaatcagatttttcagaacaaatttgagaatctatggccaaacgctagctcagtcttctaatgcaatttgtatcaaagattccttcttcgttatcaatggaaaagcatttgtttttcttatttatacattttaattttcacagtgaa

SEQ ID NO: 8 - DNA, 346 bp, Cauliflower mosaic virus
  source 1..346: mol_type = other DNA
tgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaaca

SEQ ID NO: 9 - DNA, 633 bp, Gossypium hirsutum
  source 1..633: mol_type = genomic DNA
acaacttttctctaccaatcaaatttaaaaaatagaaaaatgaaaatcgatgaattggatcaccacaatttagcccaaagaaaaacacagtcaacccctctcacagggtaggaatgatttcgaggtatagatagacatagtaacgggcaactttaactattgctgcctcgatttgaggaaaatatcaaatccaagacaaaaatttcaattatacactatgccataccattataaatatccccgttcgcaatatcatcaccattatttgaatttgcattgcaacattcgtcaccgttagttataccatcaccatcacttaattactaaaataattattggtttctcaatatgaaaaagctcgagtgcattttcttttgaatatcaaccgaaaagaaaggaaaaactaaagattttggaaggtgacggggaaaccaaaaaggaaattttgggcatttttaaaatgagaaagacgaatgtaataacccatttttctttcttactctgacaacgccacagatgctttacatgcatcatgtgatcgtgggggacccgaaacttggcatacggaaagcaccaacggcacagcattaaaagaaattgtgtataatgttaaaagaccattaattcagtctc

SEQ ID NO: 10 - DNA, 1005 bp, Gossypium hirsutum
  source 1..1005: mol_type = genomic DNA
acaacttttctctaccaatcaaatttaaaaaatagaaaaatgaaaatcgatgaattggatcaccacaatttagcccaaagaaaaacacagtcaacccctctcacagggtaggaatgatttcgaggtatagatagacatagtaacgggcaactttaactattgctgcctcgatttgaggaaaatatcaaatccaagacaaaaatttcaattatacactatgccataccattataaatatccccgttcgcaatatcatcaccattatttgaatttgcattgcaacattcgtcaccgttagttataccatcaccatcacttaattactaaaataattattggtttctcaatatgaaaaagctcgagtgcattttcttttgaatatcaaccgaaaagaaaggaaaaactaaagattttggaaggtgacggggaaaccaaaaaggaaattttgggcatttttaaaatgagaaagacgaatgtaataacccatttttctttcttactctgacaacgccacagatgctttacatgcatcatgtgatcgtgggggacccgaaacttggcatacggaaagcaccaacggcacagcattaaaagaaattgtgtataatgttaaaagaccattaattcagtctcatccaaccacgcttaaaagtcttcatgccttttctcacctctgatttcatctaatgaaaagcggacaagttgaaggatcactcgttgcttgtgtgagctttcattatttattattatgttttaggtaaccataggaagaagccattaacaacagcatgaaaaacagctagtttctccgcaaacaagataaacttttatactttttaccactgcacccccccccaaagaccagtttttaactccacctaccaagcattcaagaagcaccaaccaacttaattaccagcttaacaagacagtacaggtttctgggatatttgtagtctctcaaggacatcaccacctccactcaccttcccatttttctctagc

(19) United States
(12) Patent Application Publication
(10) Pub. No.: US 2025/0389055 A1
(43) Pub. Date: Dec. 25, 2025

(54) VITRO LOW-TRASH COTTON FIBER PRODUCTION

(71) Applicant: Galy Co., Boston, MA (US)

(72) Inventor: Luciano Luiz Bueno, Boston, MA (US)

(73) Assignee: Galy Co., Boston, MA (US)

(21) Appl. No.: 19/317,650

(22) Filed: Sep. 3, 2025

Related U.S. Application Data
(63) Continuation of application No. PCT/US2024/017982, filed on Mar. 1, 2024.
(60) Provisional application No. 63/449,743, filed on Mar. 3, 2023.

(51) Int. Cl. D01C 1/00; A01H 4/00; A01H 6/60

(52) U.S. Cl. CPC D01C 1/00; A01H 4/00; A01H 6/604

(57) ABSTRACT

A composition comprising harvested cotton fiber from cotton cells elongated in a bioreactor, wherein the cotton fiber has a trash content of less than 250 Count/gram (Cnt/g) dry mass is disclosed. Also disclosed are in vitro methods for producing cotton fiber with a low trash content at harvest.

### CROSS-REFERENCE TO RELATED APPLICATIONS

This application is a continuation of Patent Cooperation Treaty Application Serial No. PCT/US2024/017982, filed on Mar. 1, 2024, and titled “IN VITRO LOW-TRASH COTTON FIBER PRODUCTION,” which is incorporated by referenced herein in its entirety and which claims the benefit of priority of U.S. Provisional Patent Application Ser. No. 63/449,743, filed on Mar. 3, 2023, and titled “IN VITRO LOW-TRASH COTTON FIBER PRODUCTION,” which is incorporated by reference herein in its entirety.

### REFERENCE TO SEQUENCE LISTING

This specification includes a sequence listing submitted herewith, which includes the file entitled GALY-68865WO.xml having the following size: 37,406 bytes which was created Feb. 23, 2024, the contents of which are incorporated by reference herein.

### BACKGROUND

Cotton is the most widespread non-food crop in the world. However, cotton production is expensive both in terms of money and resources required for its successful cultivation. For example, cotton is a water-intensive crop, with an estimated 9,000-17,000 liters of water required for each kilogram of cotton fiber produced. This equates to enough drinking water to sustain 5,000 people for a day to produce enough cotton to make two t-shirts. Similarly, cotton cultivation requires land, which must be otherwise diverted from other crop production, such as food production. It is estimated that for every acre of cotton grown, only about 500 kilograms of cotton fiber is produced. Cotton cultivation is also a net-emitter of greenhouse gasses, with approximately between 0.75 and 2.25 kilograms of carbon dioxide gas emitted per kilogram of cotton fiber produced. Moreover, because cotton is a plant, its cultivation can lead to failed crops, mistimed crops, and even excess production. Every year, billions of dollars are spent on logistics to overcome unexpected cotton harvest results.

Compounding the environmental costs associated with cotton production are losses in fiber quantity and quality incurred during the several steps required to create useable, high-quality cotton fibers starting from unharvested plants in a field. As a natural product, cotton fibers may vary in fiber quality, which affects finished-product quality and thus potential use and value of the cotton as a commodity. High quality cotton fiber is characterized as white in color, with long and fine fibers of high strength.

The initial steps prior to spinning cotton, e.g., harvesting, opening, cleaning, and carding, are particularly critical in determining the quality and quantity, of the final, usable product. For example, mechanical harvesting drastically increases productivity and lowers costs. However, it leads to more fiber loss and damage when compared to more labor-intensive harvesting methods. In particular, mechanical harvesting increases the trash content in the harvested cotton. Trash content refers to non-fiber particles (e.g., leaf particles, dirt, dust, ash, and other foreign matter) in the harvested cotton. If not reduced or removed, the trash content in cotton fiber will degrade yarn evenness, strength, and appearance in the final product. Consequently, the higher the trash content, the more processing required to clean the cotton. Unfortunately, the steps required to remove a high trash content not only increases costs, but also introduces fiber loss and degradation. It has been found, for example, that the cleaning steps increase the amount of undesired short fibers and neps in the cotton.

Thus, beyond consideration of the environmental impacts, the steps and methods used to grow, harvest, and process cotton have always required balance and compromises to yield the most profitable end-product.

In vitro production of plant cell compositions can overcome a number of limiting factors associated with in planta production of plant-derived products, thereby providing a reliable, energy-efficient, and eco-friendly alternative to traditional agriculture. For example, plant cell compositions produced in vitro can be continuously available, while crops grown in planta are often subject to a cyclic availability. Moreover, crops grown in vitro are not exposed to the environment of a dirty field or harvesting, both of which introduce trash content into the cotton fiber.

Accordingly, there is a need for in vitro methods of producing cotton.

### SUMMARY OF THE DISCLOSURE

The present invention provides methods and compositions for the in vitro production of cotton fiber. The methods and compositions of the invention can be scaled up, thereby allowing industrial scale production of cotton fiber. By using these methods of in vitro cotton production, cotton fiber with an extremely low trash content is produced for harvest, which does not require intense cleaning steps that would otherwise degrade the fiber. By producing cotton fiber from cells in an isolated environment, the claimed methods are able to produce cotton fiber that has not been exposed to the contaminants of an outdoor farm, e.g., dirt, ash, and other particulates. Moreover, because the cotton is produced from only cells, the fibers are exposed to less non-fiber plant materials, e.g., pieces of leaves, seeds, and stems of a cotton plant which simply do not exist using the in vitro methods of the invention. These contaminants and parts of cotton plants make up the vast majority of trash content in cotton fiber produced using traditional in planta methods. The claimed methods isolate the growing cotton cells from these possible sources of trash contamination, and are thus able to produce, at harvest, cotton with an extremely low non-fiber content. Further the methods of the invention produce cotton fiber with a low trash count by weight, but also a low trash count when measured as a counter of individual non-lint particles.

Moreover, this cotton can be produced using approximately 77% less water, 80% less land, and producing approximately 84% less carbon dioxide emissions than traditional in planta methods. Despite the lower resource costs, the methods of the invention produce cotton fiber much faster that in planta methods. Whereas cotton traditionally requires 5-6 months from planting to harvest, the in vitro methods of the present disclosure can lead to a cotton fiber harvest in approximately 45 days. Additionally, because the disclosed methods are in vitro as opposed to in planta, they can be more rigidly controlled. Therefore, the propensity for failed, mistimed, or excess crops can be reduced, if not completely, eliminated.

Surprisingly, the Inventors of the present invention overcame many of the obstacles associated with in vitro crop production. The Inventors discovered that, unexpectedly, nearly all tissues of a cotton explant can be used to produce a proliferating cell aggregate to inoculate a bioreactor for in vitro cotton production. Moreover, the proliferating cell aggregates can be stably cold-stored. Additionally, a bioreactor inoculated with the proliferating cell aggregates can quickly leads to cell doubling, and the doubled cells can be elongated for cotton fiber production.

Preferably, the methods for producing cotton fiber may include inoculating the bioreactor or a culture with cotton ovule cells. The methods may alternatively or additionally include inoculating the bioreactor or culture with cells from a proliferating cell aggregate. Preferably, the proliferating cell aggregate is a friable callus.

Another insight of the present invention is that the nep content of the harvested content can be reduced in the present methods by removing cotton ovules from the culture after elongation of the cotton cells. By reducing the nep content in the cotton at harvest, the resulting fibers are of higher quality and require less processing. In certain aspects, cotton ovules may be identified in, and thus able to be removed from, a culture after cotton cell elongation by looking for clusters of non-elongated cells. This not only reduces the nep content, but also improves the already-low trash content of the cotton fiber produced using the in vitro methods of the invention.

Thus, the present invention includes a method for producing cotton fiber. In an exemplary method, the method includes inoculating a bioreactor with cotton cells; multiplying the cells in the bioreactor; elongating the multiplied cells; and harvesting cotton fiber from the elongated cells, wherein the harvested cotton has a low trash content.

In preferred aspects, the cotton has a trash content of at most 25 Cnt/g (number of individual non-lint particles per gram of harvested cotton fiber). In certain aspects, the cotton has a trash content of between about 25 Cnt/g and about 50 Cnt/g. In certain aspects, the cotton has a trash content of between about 50 Cnt/g and about 75 Cnt/g. In certain aspects, the cotton has a trash content of between about 75 Cnt/g and about 100 Cnt/g. In certain aspects, the cotton has a trash content of between about 100 Cnt/g and about 125 Cnt/g. In certain aspects, the cotton has a trash content of between about 125 Cnt/g and about 150 Cnt/g. In certain aspects, the cotton has a trash content of between about 150 Cnt/g and about 175 Cnt/g. In certain aspects, the cotton has a trash content of between about 175 Cnt/g and about 200 Cnt/g. In certain aspects, the cotton has a trash content of between about 200 Cnt/g and about 225 Cnt/g. In certain aspects, the cotton has a trash content of between about 225 Cnt/g and about 250 Cnt/g. In preferred aspects, cotton produced and harvested using methods of the invention has a trash content of less than 250 Cnt/g.

The methods of the present invention can be used to produce at least 1 kilogram of cotton fiber for every 4,000 liters of water used in the method. In some instances, the methods of the invention can produce at least 1 kilogram of cotton fiber for between every 2,000 and 4,000 of water used in the method.

In certain aspects, the methods may further include obtaining cells from a cotton explant; and contacting the cells from the cotton explant with a callus induction medium to produce the friable callus. Surprisingly, the cells from a cotton explant may be from cotton apical meristems, cotyledons, young leaves, hypocotyls, ovules, stems, mature leaves, flower, flower stalks, floral whorls, roots, bulbs, germinated seeds, somatic and zygotic embryo, and/or cambial meristematic cells (CMC).

The methods may further include dissociating cells from the friable callus; and culturing the dissociated cells. The cultured dissociated cells may be used to inoculate the bioreactor. Culturing the dissociated cells may include culturing the dissociated cells in a liquid or semi-solid medium to form a cell suspension. The methods may also include cry opreserving the cell suspension; and inoculating the bioreactor with the cryopreserved cell suspension, the method further comprises homogenizing the cell suspension to form a fine cell suspension. Homogenizing may include one or more of subculturing the cell suspension; filtering the cell suspension; pipetting and/or decanting the cell suspension; and adding pectinase to the suspension.

The methods may also include separating the elongated cells from any non-elongated cells; and harvesting cotton fiber from the separated elongated cells. In addition, the method may include recycling any non-elongated cells for use in subsequent iterations of the method.

The present invention also provides compositions comprising harvested cotton fiber from cotton cells elongated in a bioreactor, wherein said cotton fiber has a trash content of less than 250 Count/gram (Cnt/g) dry mass.

In exemplary compositions of the invention, the harvested cotton fiber has a trash content of between about 25 Cnt/g dry weight and about 50 Cnt/g dry weight; 50 Cnt/g dry weight and about 75 Cnt/g dry weight; 75 Cnt/g dry weight and about 100 Cnt/g dry weight; 100 Cnt/g dry weight and about 125 Cnt/g dry weight; 125 Cnt/g dry weight and about 150 Cnt/g dry weight; 150 Cnt/g dry weight and about 175 Cnt/g dry weight; 175 Cnt/g dry weight and about 200 Cnt/g dry weight; 200 Cnt/g dry weight and about 225 Cnt/g dry weight; or 225 Cnt/g dry weight and about 250 Cnt/g dry weight.

In certain compositions, prior to harvest cotton ovule cells are removed from the composition.

In an exemplary composition, the harvested cotton has a seed coat nep content of between about 15 Cnt/g and about 90 Cnt/g. In certain aspects, compositions of the invention include harvested cotton that comprises at most 3% by dry weight of a trash content. In preferred aspects, the harvested cotton comprises at most 1% by dry weight of a trash content. Preferably, the harvested cotton comprises at most 8.5% by dry weigh a short fiber content (SFC). In certain aspects, the harvested cotton comprises an immature fiber content of at most about 10%. In more preferred aspects, the harvested cotton comprises an immature fiber content of at most about 8%.

In some compositions of the invention, said cotton fibers have a length uniformity of at least 70%. Preferably, said cotton fibers have an average thickness of a secondary wall of at least 4 microns (pm). In certain aspects, said cotton fibers comprise, by dry weight, 88% to 96% cellulose, 1.1% to 1.9% protein, and 0.7% to 1.2% pectic substance. In some compositions of the invention, said cotton fibers further comprise, by dry weight, 0.7% to 1.6% ash, 0.4% to 1.0% wax, 0.1% to 1.0% sugar, and 0.5% to 1.0% organic acid. Preferably, said cellulose comprise at least 80% by dry weight crystalline cellulose as measured by X-ray diffraction.

Generally, two species of cotton are commercially cultivated throughout the world, namely Gossypium barbadense (G. barbadense) and Gossypium hirsutum (G. hirsutum). Traditionally, cotton cultivars of G. barbadense produce relatively long fibers, which may be referred to as extra-long staple (ELS) cotton fibers. G. hirsutum cultivars typically produce comparatively relatively short fibers, which are often referred to as Upland cotton fibers. Other regions around the world produce ELS cottons, which may possess unique fiber qualities, such as Egyptian Pima and Indian Pima. ELS cotton is generally considered a premium material of higher quality and therefore produces higher value products. Types of ELS cotton include, for example, American Pima, Egyptian, and Indian Suvin.

Isolation and/or cross breeding of cotton species has spawned genetic variations in cultivars from different regions. Though most ELS cotton cultivars belong to the species G. barbadense, ELS cotton cultivars from certain regions, such as American Pima, may possess superior fiber qualities compared to ELS cotton cultivars grown in other regions of the world. ELS cotton cultivars usually command a premium over G. hirsutum fibers (Upland).

Accordingly, in certain aspects, methods and compositions of the invention include the use of cotton cells to inoculate a bioreactor are from and/or derived from a cotton plant of the Gossypium species selected from G. hirsutum, G. arboreum, G. barbadense, G. anomalum, G. armourianum, G. klolzchianum, G. raimondii, G. herbaceum, or a progeny or a hybrid of any thereof. In certain preferred embodiments, the species is G. hirustum (upland cotton). In other preferred embodiments, the species is G. barbadense or a hybrid thereof.

In certain aspects, the cotton cells are from and/or derived from an Egyptian cotton cultivar. In certain aspects, the cotton cells are from and/or derived from a Pima (American Egyptian) cultivar.

In certain preferred aspects, the cotton cells are from or derived from organic cotton cells, which include no introduced genetic modifications. Advantageously, the isolated nature of the presently disclosed methods can be used to assure that methods using organic cotton cultivars are produced using organic-only reagents.

Exemplary embodiments include the following:

Embodiment 1: A composition comprising harvested cotton fiber from cotton cells elongated in a bioreactor, wherein said cotton fiber has a trash content of less than 250 Count/gram (Cnt/g) dry mass.

Embodiment 2: The composition of embodiment 1, wherein the harvested cotton fiber has a trash content of between about 25 Cnt/g dry weight and about 50 Cnt/g dry weight; 50 Cnt/g dry weight and about 75 Cnt/g dry weight; 75 Cnt/g dry weight and about 100 Cnt/g dry weight; 100 Cnt/g dry weight and about 125 Cnt/g dry weight; 125 Cnt/g dry weight and about 150 Cnt/g dry weight; 150 Cnt/g dry weight and about 175 Cnt/g dry weight; 175 Cnt/g dry weight and about 200 Cnt/g dry weight; 200 Cnt/g dry weight and about 225 Cnt/g dry weight; or 225 Cnt/g dry weight and about 250 Cnt/g dry weight.

Embodiment 3: The composition of embodiment 1 or embodiment 2, wherein prior to harvest cotton ovule cells are removed from the composition.

Embodiment 4: The composition of any one of embodiments 1-3, wherein the harvested cotton has a seed coat nep content of between about 15 Cnt/g and about 90 Cnt/g-

Embodiment 5: The composition of any one of embodiments 1-4, wherein the harvested cotton comprises at most 3% by dry weight of a trash content.

Embodiment 6: The composition of embodiment 5, wherein the harvested cotton comprises at most 1% by dry weight of a trash content.

Embodiment 7: The composition of any one of embodiments 1-6, wherein the harvested cotton comprises at most 8.5% by dry weigh a short fiber content (SFC).

Embodiment 8: The composition of any one of embodiments 1-7, wherein the harvested cotton comprises an immature fiber content of at most about 10%.

Embodiment 9: The composition of embodiment 8, wherein the harvested cotton comprises an immature fiber content of at most about 8%.

Embodiment 10: The composition of any one of embodiments 1-9, wherein said cotton fibers have a length uniformity of at least 70%.

Embodiment 11: The composition of any one of embodiments 1-10, wherein said cotton fibers have an average thickness of a secondary wall of at least 4 microns (pm).

Embodiment 12: The composition of any one of embodiments 1-11, wherein said cotton fibers comprise, by dry weight, 88% to 96% cellulose, 1.1% to 1.9% protein, and 0.7% to 1.2% pectic substance.

Embodiment 13: The composition of embodiment 12, wherein said cotton fibers further comprise, by dry weight, 0.7% to 1.6% ash, 0.4% to 1.0% wax, 0.1% to 1.0% sugar, and 0.5% to 1.0% organic acid.

Embodiment 14: The composition of embodiment 12 or embodiment 13, wherein said cellulose comprise at least 80% by dry weight crystalline cellulose as measured by X-ray diffraction.

Embodiment 15: A method for producing cotton fiber, the method comprising: inoculating a bioreactor with cotton cells; multiplying the cells in the bioreactor; elongating the multiplied cells; and harvesting cotton fiber from the elongated cells, wherein the harvested cotton fiber has a trash content of less than 250 Count/gram (Cnt/g) dry mass.

Embodiment 16: The method of embodiment 15, wherein the harvested cotton fiber has a trash content of between about 25 Cnt/g dry weight and about 50 Cnt/g dry weight; 50 Cnt/g dry weight and about 75 Cnt/g dry weight; 75 Cnt/g dry weight and about 100 Cnt/g dry weight; 100 Cnt/g dry weight and about 125 Cnt/g dry weight; 125 Cnt/g dry weight and about 150 Cnt/g dry weight; 150 Cnt/g dry weight and about 175 Cnt/g dry weight; 175 Cnt/g dry weight and about 200 Cnt/g dry weight; 200 Cnt/g dry weight and about 225 Cnt/g dry weight; or 225 Cnt/g dry weight and about 250 Cnt/g dry weight.

Embodiment 17: The method of embodiment 15 or embodiment 16, wherein the bioreactor is inoculated with cotton cells comprising cotton ovule cells.

Embodiment 18: The method of any one of embodiments 15-17, further comprising identifying and removing non-elongated cells prior to harvesting the cotton fiber, thereby removing cotton ovules from the bioreactor.

Embodiment 19: The method of any one of embodiments 15-18, wherein the method produces at least 1 kilogram of cotton fiber for every 4,000 liters of water used in the method.

Embodiment 20: The method of embodiment 19, wherein the method produces at least 1 kilogram of cotton fiber for between every 2,000 and 4,000 of water used in the method.

Embodiment 21: The method of any one of embodiments 17-20, wherein the bioreactor is inoculated with cells from a proliferating cell aggregate.

Embodiment 22: The method of embodiment 21, wherein the proliferating cell aggregate is a friable callus.

Embodiment 23: The method of embodiment 22, further comprising: obtaining cells from a cotton explant; and contacting the cells from the cotton explant with a callus induction medium to produce the friable callus.

Embodiment 24: The method of embodiment 23, wherein the cells from a cotton explant are from cotton apical meristems, cotyledons, young leaves, hypocotyls, ovules, stems, mature leaves, flower, flower stalks, floral whorls, roots, bulbs, germinated seeds, somatic and zygotic embryo, and/or cambial meristematic cells (CMC).

Embodiment 25: The method of any one of embodiments 22-24, wherein the method further comprises: dissociating cells from the friable callus; and culturing the dissociated cells.

Embodiment 26: The method of embodiment 25, wherein the method further comprises inoculating the bioreactor with the cultured dissociated cells.

Embodiment 27: The method of embodiment 25 or embodiment 26, wherein culturing the dissociated cells comprises culturing the dissociated cells in a liquid or semi-solid medium to form a cell suspension.

Embodiment 28: The method of embodiment 27, wherein the method further comprises: cryopreserving the cell suspension; and inoculating the bioreactor with the cryopreserved cell suspension.

Embodiment 29: The method of embodiment 27 or embodiment 28, wherein the method further comprises homogenizing the cell suspension to form a fine cell suspension.

Embodiment 30: The method of embodiment 29, wherein homogenizing includes one or more of: subculturing the cell suspension; filtering the cell suspension; pipetting and/or decanting the cell suspension; and adding pectinase to the suspension.

Embodiment 31: The method of any one of embodiments 15-30, further comprising: separating the elongated cells from any non-elongated cells; and harvesting cotton fiber from the separated elongated cells.

Embodiment 32: The method of embodiment 31, further comprising recycling any non-elongated cells for use in subsequent iterations of the method.

Embodiment 33: The method of any one of embodiments 15-32, further comprising: maturing the elongated multiplied cells using a maturation medium; and harvesting cotton fiber from the mature elongated cells.

Embodiment 34: The method of any one of embodiments 15-33, wherein the harvesting comprises: separating cotton fiber from the maturation medium; and passing air through the separated cotton fibers until the fibers have a moisture content of less than 5%.

Embodiment 35: The method of any one of embodiments 15-34, wherein the harvested cotton has a seed coat nep content of between about 15 Cnt/g and about 90 Cnt/g-

Embodiment 36: The method of any one of embodiments 15-35, the method further comprising a step of bleaching the cotton fiber.

Embodiment 37: The method of embodiment 36, wherein the step of bleaching the cotton fiber occurs prior to harvesting the cotton fiber.

Embodiment 38: The composition of any one of embodiments 1-14, wherein the cotton cells are organic cotton cells.

Embodiment 39: The composition of any one of embodiments 1-14 or embodiment 38, wherein the cotton cells are from and/or derived from a cotton plant of the Gossypium species selected from G. hirsuliim. G. arboreum. G. barbadense. G. anomalum. G. armourianum, G. klolzchianum, G. raimondii, G. herbaceum, or a progeny or a hybrid of any thereof.

Embodiment 40: The composition of embodiment 39, wherein the species is G. hirustum (upland cotton).

Embodiment 41: The composition of embodiment 39, wherein the species is G. barbadense or a hybrid thereof.

Embodiment 42: The composition of embodiment 41, wherein the cotton cells are from and/or derived from an Egyptian cultivar.

Embodiment 43: The composition of embodiment 41, wherein the cotton cells are from and/or derived from a Pima (American Engyptian) cultivar.

Embodiment 44: The method of any one of embodiments 15-37, wherein the cotton cells are from and/or derived from a cotton plant of the Gossypium species selected from G. hirsutum, G. arboreum, G. barbadense, G. anomalum, G. armourianum, G. klotzchianum, G. raimondii, G. herbaceum, or a progeny or a hybrid of any thereof.

Embodiment 45: The method of embodiment 44, wherein the species is G. hirustum (upland cotton).

Embodiment 46. The method of embodiment 44, wherein the species is G. barbadense or a hybrid thereof.

Embodiment 47: The method of embodiment 46, wherein the cotton cells are from and/or derived from an Egyptian cultivar.

Embodiment 48: The method of embodiment 46, wherein the cotton cells are from and/or derived from a Pima (American Egyptian) cultivar.

### BRIEF DESCRIPTION OF THE DRAWINGS

FIG. 1 shows an exemplary method of the invention

FIG. 2 shows a flowchart of the concept of a commercial scale process for the cotton fiber in vitro production.

FIG. 3 shows a computer system that is programmed or otherwise configured to implement methods provided herein.

### DETAILED DESCRIPTION

The present invention provides methods and compositions for the in vitro production of cotton fiber that has a low trash content, when compared with cotton grown in a field using modern farming techniques. The methods of the disclosure are cell-based, and do not require growing cotton plants for the cultivation of cotton fiber. Instead, these methods allow quick and efficient cultivation of cotton fiber in a controlled environment.

Advantageously, the methods and compositions of the invention can be scaled up, thereby allowing industrial scale production of cotton fiber. By using these methods of in vitro cotton production, cotton fiber can be produced using approximately 77% less water and 80% less land than traditional in planta methods. The methods can also produce cotton fiber harvest that results in about 84% less carbon dioxide emissions when compared with traditional methods. Despite the lower resource costs, the methods of the invention produce cotton fiber much faster that in planta methods. Whereas cotton traditionally requires 5-6 months from planting to harvest, the in vitro methods of the present disclosure can lead to a cotton fiber harvest in approximately 45 days.

The presently disclosed, in vitro methods produce cotton fiber with an extremely low trash content at harvest. As a natural product, cotton fiber is variable in quality. Thus, cotton is classified by its fiber quality, usually using High Volume Instrument (HVI) classification. Fiber quality is a combination of parameters, which include fiber length, uniformity, strength, micronaire, color, and trash content. The combined measures of fiber quality are ultimately used to set the market value for a cotton fiber harvest. Among the parameters of cotton fiber quality, the trash content is one of the most critical.

Trash, in general, refers to non-lint content contained within cotton fiber. Trash may include undeveloped seeds, seedcoat fragments, particles of leaf, dust, sand, ash, stems, and other foreign materials. High quantities of trash in cotton fiber reduces its processing performance, yarn yield, and final quality of fabrics. Consequently, the U.S. Cotton Futures Act includes foreign matter content of cotton fiber as a parameter for assigning cotton a grade.

When cotton harvest transitioned from a labor-intensive hand-picking system to a mechanical harvest approach, the mass of cotton picked per unit time increased. Unfortunately, the amount of trash that was captured together with the harvest also increased machine harvested lint has on average about 10-30% more trash content than handpicked cotton fiber. It has also been found that machine harvesting not only increases the trash content of harvested content, but also changes the profile of the trash content. Typically, the trash content of handpicked cotton is comprised of larger particles, which tend to be easily removed from cotton lint during cleaning. However, machine harvested cotton often includes trash contents made from smaller particles, which have a tendency to stain the lint fiber and be far more difficult to remove from the cotton fiber. Consequently, due to its higher trash content and corresponding discoloration, machine harvested cotton generally has a fiber quality two grades below that of handpicked cotton.

Further, because machine harvested cotton fiber tends to have a higher trash content, it requires more processing and cleaning to improve its HVI classification/fiber quality grade. However, cleaning and processing cotton to remove excess trash content comes at costs. First, in terms of pure economics, the steps and time needed to clean the cotton all add to increased processing costs. Further, cleaning and processing damages cotton fibers, and may for example, decrease fiber strength and average length and/or increase short fiber content. Moreover, cleaning and processing steps also produce lost cotton fiber.

In fact, machine harvesting and processing for cotton abounds with inefficiencies. It is estimated that about 2-10% of cotton fiber falls to the ground in the field before harvest. Machine harvesting is also estimated to leave about 5-15% of cotton fiber in the field due to ground loss or cotton bolls remaining on their stems.

The in vitro methods for producing cotton fiber of the present invention eliminate these sources of inefficiency. The methods of the invention produce cotton that is incredibly clean at harvest—generally having a trash of at most about 25, 30, 40, 50, 60, 70, 80, 90, 100, 110, 120, 130, 140, 150, 160, 170, 180, 190, 200, 220, 230, 240, or about 250 Cnt/g (number of individual non-lint particles per gram of harvested cotton fiber).

The claimed methods are able to produce this cotton fiber, wholly from cells in a bioreactor, which helps isolate or eliminate the cotton fiber from encountering common sources of trash, e.g., stems, leaves, dirt, ash, and other biological material. Moreover, because the cotton is produced from only cells, the fibers are exposed to less non-fiber plant materials, e.g., pieces of leaves, seeds, and stems of a cotton plant—which simply do not exist using the in vitro methods of the invention. Because the cotton lint produced and harvested has such a low trash content, it requires less cleaning and processing to produce saleable cotton fiber. This both reduces costs and environmental impact relative to traditional methods. Further, producing the cotton in a controlled, enclosed environment and reducing the amount of processing/cleaning required, reduces sources of potential fiber loss endemic to traditional methods of cotton fiber production.

Additionally, because the disclosed methods are in vitro as opposed to in planta, they can be more rigidly controlled. Therefore, the propensity for failed, mistimed, or excess crops can be reduced, if not completely, eliminated. These methods can be practiced indoors, using automated machinery, and even specialized cotton cell lines to ensure a cotton fiber harvest with desired qualities. This control also assures less cotton fiber loss during harvest, which improves over the approximate 20% loss of cotton fiber during traditional, machine harvests.

Surprisingly, the Inventors of the present invention overcame many of the obstacles associated with in vitro crop production. The Inventors discovered that, unexpectedly, nearly all tissues of a cotton explant can be used to produce a proliferating cell aggregate to inoculate a bioreactor for in vitro cotton production. Moreover, the proliferating cell aggregates can be stably cold-stored. Thus, once created, cell aggregates can be used for cotton fiber production without the need to rely on living cotton plants. A bioreactor inoculated with a small number of cells from the proliferating cell aggregate quickly leads to cell doubling in the bioreactor, and the doubled cells can be elongated for cotton fiber production.

Preferably, the methods of the invention inoculate a bioreactor with cotton cells that include cotton ovules. The present inventors found that not only do the cotton ovules provide an effective way to inoculate a bioreactor, but also provide an opportunity to produce cotton fiber with improved cotton fiber qualities. Cotton fibers may include imperfections known as neps. Generally, cotton fibers include two types of neps, mechanical and biological neps. Neps affect yarn evenness, strength, and appearance of textiles made from cotton. Mechanical neps are entangled cotton fiber clusters produced during cotton fiber processing, e.g., ginning or cleaning. Biological neps are tangled fibers that include a foreign material, i.e., trash. Seed coat fragments (SCF) are the most prevalent foreign material found in neps. SCF neps account for approximately 15-30% of all neps typically found in cotton. Unfortunately, SCF neps are also among the most difficult to deal with in cotton fiber processing. They tend to include attached cotton fibers, making them more prone to create large tangles, making them more difficult to remove before the spinning process.

The present inventors discovered that adding a step of identifying and removing cotton ovules from elongated cotton cells in a bioreactor can reduce the seed coat nep content of the harvested cotton. Moreover, the present inventors discovered that these unwanted cotton ovules can be identified by the presence of clumps of elongated cells in the bioreactors, thereby facilitating their removal.

In certain aspects, methods of the invention may include a step of bleaching the cotton fiber. As a natural fiber, cotton often contains discoloration, especially in cotton with a high trash content. Often, cotton fiber undergoes a bleaching process, generally with a hydrogen peroxide and/or enzyme solution. In addition to improving coloration (i.e., whiteness), bleaching may also improve cotton fiber dye absorbency and levelness. Further, the present Inventors discovered that a bleaching step, when used with the present methods, may further reduce the seed coat nep content of the harvested cotton. In certain aspects, this step of bleaching may occur before the cotton fiber is harvested.

Another improvement conferred by the present methods is the interaction of the cotton fiber and common bleaching agents. Typically, when bleaching cotton, the amount of bleaching required is determined by considering the end-use requirements of the cotton (e.g., dark or light fabric), the natural discoloration of the cotton fiber, and the trash content. When producing dyed, rather than white, end products, the consideration shifts towards the natural color and trash content. Hydrogen peroxide liquors in conjunction with heat are the primary means for bleaching cotton fibers. However, metals and other impurities, such as iron, copper, and manganese, when introduced into the cotton fiber as trash, may cause a catalytic decomposition of the hydrogen peroxide, which reduces bleach efficacy and leads to fiber damage. Similarly, despite bleaching, some types of trash from soil that persist in cotton fiber may cause yellowing of finished product. The presently disclosed methods greatly reduce these sources of contamination, making the cotton fiber more amenable to bleaching steps. As explained, not only is bleaching commonly used to improve cotton fiber color, but the present Inventors discovered that it reduces the already low seed coat nep content of the harvested cotton fiber.

In certain aspects, the present invention provides methods and compositions for the in vitro production of cotton fiber using cotton cells that express selected genes of interest. Due to the ubiquity of cotton production around the world, there has long been interest in modifying cotton plants, through genetic alterations or selective breeding, in order to produce cotton plants with commercially-relevant characteristics. This may lead to cotton plants that include traits that result in better growth under certain agronomic conditions, pest and disease resistance, and increased yield. Cotton plants have also been modified to improve cotton fiber-related traits. For example, cotton plants have been developed with increased fiber length, strength, amenability to dye, and decreased fuzz fiber, immature fiber content, micronaire, fiber uniformity, and fiber maturity ratio. The present invention includes, methods and compositions in which cotton cells that express selected genes, which lead to the aforementioned improvements, when the cells are used in methods of in vitro cotton fiber production. Moreover, due to the unique nature of the disclosed methods, the selected genes may provide improvements uniquely tailored to in vitro cotton fiber production.

The methods of the disclosure can be cell-based, and not require the growth of entire cotton plants for modifying cotton cells to express selected genes of interest and/or cultivation of cotton fiber. These methods allow quick and efficient cultivation of cotton fiber in a controlled environment. Cotton cells expressing the selected genes may show improved in vitro cotton fiber cell development compared to wildtype control cells. For example, the selected genes may improve in vitro fiber production by increasing the speed of cotton fiber cell development and/or increase the quantity cotton fiber cells. The selected genes can also lead to unexpectedly quick cell growth, fast cell multiplication/duplication, early cotton fiber/pre-fiber growth, and/or efficient bioreactor inoculation.

In certain aspects, the selected genes may be introduced into cotton cells as transgenes, and the modified cotton cells used in the presently disclosed in vitro methods of cotton fiber production. Thus, the selected genes of interest can be introduced into cells and the cells used to produce cotton fiber without the need to grow cotton plants. The methods and compositions of the invention can be scaled up, thereby allowing industrial scale production of cotton fiber.

While cells derived from most, if not all, cotton varietals can be used for these in vitro methods of cotton production, some varietals possess traits that make them particularly useful for the presently disclosed methods of in vitro cotton fiber production. When used in the methods of the invention, some of these varietals show, for example, unexpectedly quick cell growth, fast cell multiplication/duplication, early cotton fiber/pre-fiber growth, and/or efficient bioreactor inoculation. In certain aspects, genes and/or gene expression patterns leading to improved in vitro cotton fiber production can be determined. Once determined, these genes and/or gene expression patterns can be introduced into other cotton plants/cells, for example, as a transgene.

The cotton cells of the invention can also be modified to include a gene selected for improvement and/or modulation of cotton fiber development in vitro, such as those disclosed by the present inventors in U.S. Provisional Application No. 63/137,952, which is incorporated by reference. These genes may improve/modulate cotton fiber development in cotton cells and/or cotton plants. However, genes that do not modulate/improve cotton fiber development in plants may nevertheless modulate/improve cotton fiber development using the in vitro methods disclosed herein. The gene selected for improvement and/or modulation of cotton fiber development may be endogenous to one or more species or varietal of cotton plant, may be a gene from, or derived from, another species, or may be a synthetic gene. The gene may be, or derived from, a cotton fiber development gene. Expression or overexpression of genes such as GhHOX3, GhHD-1, SPL5, GaMYB2, iaaM, GhPIN3a, GhWlim5, and GhFP1 has been shown to have a positive impact on cotton fiber growth (Cai, C., et al., 2018; Liu, Z. et al., 2020; Shan, C. et al., 2014; Walford, S. et al., 2012; Wang, S. et al., 2004; Zhang, M., et al., 2011; Mei, G. et al., 2019; Iqbal, A. et al., 2020).

The cotton cells of the invention can also be modified to include a developmental regulator gene, such as those disclosed by the present inventors in U.S. Provisional Application No. 63/137,952, which is incorporated by reference. In certain aspects, the cotton cells may be selected or modified to express at least one developmental regulator gene and/or at least one gene selected for improvement and/or modulation of cotton fiber development. Developmental regulator genes such as Wus2 and Bbm have been shown to have a positive impact in corn, increasing plant cell growth and transformation efficiency dramatically (Hoerster, G., 2020; Gordon-Kamm, B., 2019; Lowe, K., 2015; Lowe, K., 2018). In cotton, AtWUS was found to promote the formation of the embryogenic callus in cotton, promoting somatic embryogenesis and inducing organogenesis in cotton tissues cultured in vitro, yielding 3-4 times more embryogenic calli than the wild type control (Zheng, et al., 2014 and Bouchabke-Coussa et al., 2013). Cotton GhWUS gene was shown to have a positive impact on Arabidopsis transformation and regeneration in growth in vitro (Xiao, Y. et al., 2018). When transformed into cotton cells, such as ovule epidermal cells, these developmental growth regulators shorten the time and/or increase the number of cells that produce fiber when transformed either alone or in combination with one or more genes selected for improvement and/or modulation of cotton fiber development in vitro, such as GhHOX3, GhHD-1, SPL5, GaMYB2, iaaM, GhPIN3a, GhWlim5, and GhFP1. Tissue specific expression of developmental regulator genes Wus2 and Bbm has been shown to be essential for normal plant development in prior studies in corn (Hoerster, G., 2020; Gordon-Kamm, B., 2019; Lowe, K., 2015; Lowe, K., 2018). Therefore, tissue specific protomers were identified in cotton to ensure cotton fibers develop normally in vitro.

Exemplary promoters that can be used in accordance with the invention include cotton-specific promoters or promoter regions of endogenous cotton genes that have temporally and/or spatially regulated expression, including late embryogenesis-abundant gene DI 13 from cotton accumulates at high levels in mature seeds, leaves, embryos, and callus (Luo et al., 2008), GbPDF1, cotton PROTODERMAL FACTOR1 gene (GbPDF1) which is predominantly expressed in the epidermis of ovules and developing fibers during fiber initiation and early elongation (Deng et al., 2012), and GhMYB109, a cotton fiber specific R2R3 MYB gene (Pu et al., 2008). Similarly, transgenic reporter gene analysis has shown that a 2-kb GhMYB109 promoter was sufficient to confirm its fiber-specific expression via GUS staining. A GhSCFP promoter was shown to specifically activate transcription in seed coat and fiber associated genes.

In some cases, non-cotton specific constitutive protomers such as 35S promoter (such as CaMV 35S), an FBP7 (petunia floral binding protein 7) promoter, a tobacco TA29 promoter, would function best, in the in vitro methods of the disclosure. Exemplary 35S, FBP7, and TA29 promoter nucleic acid sequences are provided in Table 1.

One or more of the promoters used in the methods and compositions of the invention may be a PDF1 promoter, such as a GbPDF1 promoter from G. barbadense or a GhPDF1 promoter from G. hirsutum. The cotton PROTODERMAL FACTOR1 gene (PDFP) is predominantly expressed in the epidermis of ovules and developing fibers during fiber initiation and early elongation (Deng et al., 2012). In cotton plants, GbPDF1 was found to be preferentially expressed during fiber initiation and elongation, with a highest accumulation in fiber cells five days post anthesis. GbPDF1 promoter::GUS constructs in transgenic cotton predominantly expressed in the epidermis of ovules and developing fibers. (Deng et al., 2012). A 236 basepair promoter fragment of GbPDF1 was shown to promote GbPDF1 transcription in cotton. The temporally preferential expression of GbPDF1 during fiber initiation and elongation makes GbPDF1 promoters useful for controlling expression of a differently expressed gene in the in vitro methods of cotton production disclosed herein. Table 1 provides two exemplary cotton PDF1 promoters, which include the 236 base pair promoter fragment, and the full CDS of GbPDF1 and GhPDF1.

Due to the tissue and temporal preferential expression of GhACT genes in plantae, their promoter sequences are useful as promoters for the differentially expressed genes used in the in vitro methods of cotton production disclosed herein. Exemplary promoter nucleic acid sequences of GhACTI are provided in Table 1.

GhMYB109 is a cotton fiber specific R2R3 MYB gene. Reporter gene analysis showed that a 2-kb GhMYB109 promoter provided fiber-specific expression using a GUS staining construct. (Pu et al., 2008). Due to this fiber-specific expression, GhMYB109 promoters are useful as promoters for controlling expression of a differently expressed gene in the in vitro methods of cotton production disclosed herein. An exemplary GbMYB109 promoter is provided in Table 1.

G. hirsutum being fiber-specific promoter (GhSCFP) activates transcription in seed coat and fiber associated genes. (Yaqoob et al. 2020). Due to this preferential regulation of seed coat and fiber associated genes, GhSCFP promoters are useful as promoters for controlling expression of a differently expressed gene in the in vitro methods of cotton production disclosed herein. Exemplary GhSCFP promoters are provided in Table 1.

TABLE 1

| | Corresponding |
|---|---|
| Description | SEQ ID NO |
| Exemplary D113 Promoter nucleic acid sequence | SEQ ID NO: 1 |
| Exemplary GbPDF1 Promoter (GbPDF1-1) nucleic | SEQ ID NO: 2 |
| acid sequence | |
| Exemplary GbPDF1 Promoter (GbPDF1-2) nucleic | SEQ ID NO: 3 |
| acid sequence | |
| Exemplary GhMYB109 promoter nucleic acid | SEQ ID NO: 4 |
| sequence | |
| Exemplary S7 (Stunt 7) promoter nucleic acid | SEQ ID NO: 5 |
| sequence derived from subterranean clover stunt | |
| virus DNA segment 7, complete CDS (GenBank: | |
| AY159024.1) | |
| Exemplary Seed coat-specific FBP7 (floral binding | SEQ ID NO: 6 |
| protein 7) promoter nucleic acid sequence | |
| Exemplary TA29 promoter nucleic acid sequence | SEQ ID NO: 7 |
| Exemplary CaMV 35S promoter nucleic acid | SEQ ID NO: 8 |
| sequence | |
| Exemplary GhSCFP promoter nucleic acid | SEQ ID NO: 9 |
| sequence | |
| G. hirsutum cultivar Handan 5833 fiber-specific | SEQ ID NO: 10 |
| protein (SCFP) gene, including promoter region | |
| (region 1 . . . 633) and 5′ UTR (GenBank: | |
| GQ411495.1) | |

In certain aspects, the promoter includes a nucleic acid sequence having at least 90%, 95%, 96%, 97%, 98%, or at least 99% sequence identity to anyone of the genes listed in Table 1, one or more fragments thereof, or an endogenous promoter sequence for such a gene. Derivatives may include, one or more mutations, such as deletions, point mutations, restriction site alterations, nucleotide substitutions, additions and/or codon modifications. Derivatives may also include, for example, regulatory elements, and/or functional elements or modified functional elements.

Percent sequence identity refers to the percentage of identical nucleotides between two segments of a window of optimally aligned DNA. Tools and methods for alignment are well known in the art, for example, the local homology algorithm of Smith and Waterman, the homology alignment algorithm of Needleman and Wunsch, the search for similarity method of Pearson and Lipman. These algorithms may be implemented as, or included in, computer programs, for example, GAP, BESTFIT, and FASTA. An identity fraction for aligned sequence segments refers to the number of identical components that are shared by aligned test and reference sequences divided by the number of components in the reference sequence segment. Percent sequence identity is shown herein as the identity fraction multiplied by 100. The comparison of one or more DNA sequences may of an entire or full-length sequence or a portion thereof, or to a longer DNA sequence.

In certain aspects, modifying the cotton cell may involve the use of a vector. Vectors can be used to produce, transfer or manipulate transgenes, chimeric genes, and/or genetic constructs. A vector is a nucleic acid molecule, preferably a DNA molecule derived, for example, from a plasmid, bacteriophage or plant virus, into which a nucleic acid sequence may be inserted into a cotton cell plant, explant or cell. A vector may include one or more unique restriction sites, capable of autonomous replication in a selected cotton cell, tissue, explant, or plant, or integrated into the genome of a cotton cell, plant, or explant such that the cloned sequence is reproducible. The vector may be an autonomously replicating vector, which exists as an extrachromosomal entity, its replication independent of chromosomal replication, for example, a linear or closed circular plasmid, an extrachromosomal element, a mini-chromosome, or an artificial chromosome. The vector may be one which, when introduced into a cell, is integrated into the genome of the recipient cell and replicated together with the chromosome(s) into which it has been integrated.

A vector system may include a single vector or plasmid, or two or more vectors or plasmids, which contain the DNA to be introduced into the genome of a cell. A vector may also comprise a selection marker, for example, an antibiotic resistance gene that can be used for selection of suitable transformants.

In certain aspects, a vector is used to introduce genes into cotton cells. The cells may be obtained or derived from the tissue from any meristematic part of a cotton plant or explant, including apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and/or cambial meristematic cells (CMC).

Alternatively, the vector is used to introduce the gene(s)/construct(s) of the invention into a cotton plant, cotton seed, cotton explant, and/or cotton plant tissue. After introduction, cotton plants, explants, and/or cotton plant tissue can be grown. Once grown, cotton cells can be selected. In certain aspects, after introduction, cotton plants are grown for one or more generations before cells are selected. The cotton plants can be backcrossed or crossed with other cotton plants to introduce desirable genetic backgrounds before the cells are selected.

In certain aspects, cotton cells and/or plants can be modified with an induced mutation. An induced mutation is an artificially induced genetic variation, for example, using chemical, radiation or biologically-based mutagenesis. The resulting mutations may include nonsense mutations, frameshift mutations, additions deletions, insertional mutations or splice-site variants. The mutations may modulate the activity of the selected gene.

In certain aspects, the cotton cells used in the methods for producing colored cotton fiber in vitro may include the use of cotton cells that have a differently expressed gene (DEG). A differently expressed gene may have increased or decreased expression when compared to an endogenous and/or wild type gene. In certain aspects, the an endogenous and/or wild type gene may have no naturally occurring expression. A DEG may also have a different spatial and/or temporal expression pattern when compared to an endogenous and/or wild type gene. The DEG can be synthesized to include a promoter that assures the DEG is expressed at a certain level, under specific growth conditions, in certain tissues and tissue types, and/or under specified spatial and/or temporal constraints.

Cotton cells or plants of the present disclosure can be subject to a mutagenic process to give rise the DEG. This process can occur in vitro and without ever growing a whole cotton plant with the DEG.

Mutagenesis can be achieved by radiation and/or chemical means, including EMS or sodium azide treatment of seed, or gamma irradiation. Chemical mutagenesis favors nucleotide substitutions rather than deletions. Heavy ion beam (HIB) irradiation is a known technique for mutagenesis. Ion beam irradiation has two physical factors, the dose (gy) and LET (linear energy transfer, keV/um) for biological effects that determine the level of DNA damage and the size of any DNA deletion(s), and these can be adjusted according to change the extent of mutagenesis.

Biological agents can also be used to create site-specific mutations in cotton cells. These agents may include enzymes that cause double stranded breaks in DNA, which stimulate endogenous repair mechanisms. These enzymes include endonucleases, zinc finger nucleases, transposases and site-specific recombinases.

Isolation of cotton cells or plants with a selected gene may be achieved by screening mutagenized cotton plants or cells. For example, a mutagenized population of cotton plants may be screened directly for a particular genotype or indirectly by screening for a desired phenotype. Screening directly for the genotype may include assaying for the presence of mutations, e.g., using PCR- or sequencing-based assays.

In certain aspects, cotton cells are selected from cotton plants produced using the process known as TILLING (Targeting Induced Local Lesions IN Genomes). In a first step, introduced mutations are induced in a population of plants by treating seeds or pollen with a chemical or radiation mutagen, and then advancing plants to a generation where mutations will be stably inherited, typically an M2 generation where homozygotes may be identified. DNA is extracted, and seeds are stored from all members of the population to create a resource that can be accessed repeatedly over time. For a TILLING assay, PCR primers are designed to specifically amplify a single gene target of interest. PCR products from pooled DNA of multiple individuals are amplified using the primers. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease that cleaves mismatched DNA is the key to discovering novel SNPs within a TILLING population.

In some embodiments, the cotton cells, plants, and/or explants (or engineered cotton) described herein can be derived from a Gossypium species. The Gossypium species can be selected from the group consisting of G. arbor eum. G. anomalum. G. armouriamim. G. klolzchiamim. and G. raimondii. The cotton (or engineered cotton) can be derived from a Gossypium species selected from the group consisting of G. hirsulum, G. arboreum. G. bar hade use, G. anomalum, G. armourianum, G. klolzchianum, and G. raimondii. The cotton (or engineered cotton) can be Gossypium hirsulum, Gossypium barbadense, Gossypium arboretum, Gossypium herbaceum, or another species of cotton.

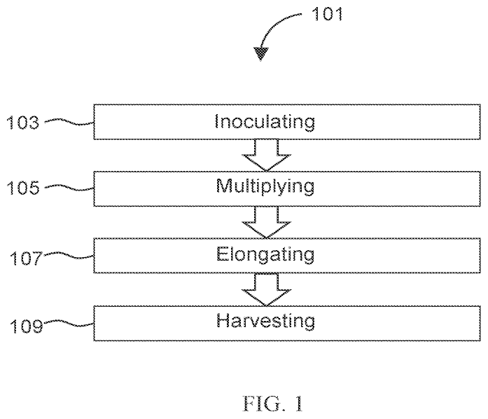

FIG. 1 provides an exemplary method 101 of the invention for the in vitro production of cotton fiber. As shown in FIG. 1, the method 101 optionally begins with modifying or selecting cotton cells 103. The cotton cells may be modified 103 to express, or selected 103 for expression, of one or more genes of interest. The cotton cells, or cells derived therefrom, can then be used to inoculate 105 a bioreactor. After inoculating 105, the method 101 requires multiplying 107 the cells in the bioreactor. The multiplied cells are then elongated 109 to produce cotton fibers. When the cotton fibers are sufficiently matured, the resulting cotton fiber is harvested 111. In certain aspects, either before or after harvesting 111, the method further includes a step of identifying and removing ovules from the cell culture. Ovules may be identified by the presence of clumps of non-elongated cells. In certain aspects, either before or after harvesting, the method further includes one or more steps of bleaching the cotton fiber and/or cotton cells, preferably, with a hydrogen peroxide liquor.

In preferred aspects, the bioreactor is inoculated 105 with a small number of cotton cells. More preferably, the cotton cells include cotton ovule cells such that the bioreactor may be inoculated with cotton ovule cells, which may include ovule epidermal cells. Generally, the bioreactor will be inoculated with cotton cells from a proliferating cell aggregate. As shown in Example 4, milligram quantities of cotton cells from a proliferating cell aggregate are sufficient to eventually inoculate a bioreactor.

Inoculating 105 may include preparing a growth medium in a vessel, such as a flask or plate, and introducing a small number of cotton cells from a proliferating cell aggregate into the medium. The vessel may then be left for inoculum growth. Alternatively, inoculum growth may occur inside the bioreactor.

The Inventors found that, surprisingly, inoculum growth under dark conditions provided superior growth. The vessel may be shaken or agitated during inoculum growth, for example, at a rate of 80-180 rpm. Preferably, inoculum growth occurs at a temperature of about 30° C. to about 35° C. Preferably, the medium is a solution that comprises plant hormones, plant growth regulators, and/or sucrose and/or glucose. Inoculum growth generally takes about 16 days, but may be more or less as desired or due to conditions or individual cotton cell lines.

The inoculum may be a cell suspension in a liquid or semi-solid medium. The suspension may be optionally homogenized to provide a fine cell suspension culture. The present Inventors discovered that a homogenous cell suspension can provide more reproducible and reliable results when inoculating a bioreactor.

Homogenizing may include any methods known in the art, including one or more of subculturing the suspension, filtering, pipetting/decanting, and/or addition of a low concentration of pectinase.

The resulting inoculum is then introduced into a bioreactor. Alternatively, the resulting inoculum can be preserved, e.g., by freezing, for later use in inoculating a bioreactor. The inoculum or homogenous cell suspension, which includes cells that include one or more selected genes of interest, may be cryopreserved indefinitely, for example, in liquid nitrogen. This generally requires suspending cells from the inoculum/homogenous cell suspension in a cryoprotectant solution, for example a solution of glycerol and sucrose. The cryoprotectant solution can be supplement, for example, using proline. Cryopreserved cells can be recovered, for example, using a recovery media, before their use in inoculating a bioreactor.

The proliferating cell aggregate may be a callus. Preferably, the proliferating cell aggregate is a friable callus, which is not sticky or soft, but is also not so hard or dense that it cannot be physically broken or crumbled. A friable callus thus differs “a hard callus”, which is compact and brittle, and thus not amenable to being broken or crumbled. The Inventors discovered that a friable callus allows for simple mechanical manipulation to easily disassociate individual cells from the friable callus for use in inoculating 105 a bioreactor and/or preparing an inoculum.

After inoculating 105, the method 101 requires multiplying 107 the cells in the bioreactor. This phase generally lasts for between 5 and 12 days, with duplication for the cotton cells taking approximately 1 to 3 days depending on cotton lineage. The cells may be duplicated, for example, by culturing the cells in a cell culture medium.

The multiplied cells are then elongated 109 to produce cotton fibers. This may include using an elongation medium to induce elongation in the multiplied cells. In certain aspects, the elongation medium facilitates release of a phenolic compound from a vacuole of an elongated cotton cell. The elongated cells may include cotton pre-fibers, which will mature into cotton fibers.

In certain aspects, a semi-solid elongation medium is used to elongate the cotton cells. The Inventors made the surprising discovery that superior results are achieved when using a semi-solid medium as opposed to a liquid medium.

Optionally, after elongation, the elongated cotton cells are separated from any nonelongated cotton cells. The non-elongated cotton cells will not mature into cotton fibers. However, they may be recycled and used in subsequent iterations of the method. Separating the elongated cotton cells from the non-elongated cells may include one or more of filtering, sieving, decanting, and centrifuging the cells.

Once separated, the elongated cotton cells, which at this point may have cotton prefibers, are matured. Maturing the cells may include the use of a maturation medium. During maturation, sugars are combined in the cells to produce cellulose, which is the main component of cotton fiber (natural glucose polymerization) that occurs inside the cell forming a secondary wall. The cotton pre-fibers increase in number, density, and/or length.

After maturation, cotton fiber harvested 111 from the cotton cells, by for example, separating the fibers from the cells in a solution/buffer. The harvested cotton fiber is then dried to a moisture content of less than 5% by, for example, passing air through the cotton fiber.

Thus, the method 101 can produce cotton fibers from cotton cells without growing cotton plants. These methods allow quick and efficient cultivation of cotton fiber in a controlled environment.

The method 101 may also include preparing a friable callus. A friable callus can be made, for example, by obtaining cells from a cotton explant and contacting the cells with a callus induction medium. Surprisingly, the Inventors discovered that tissue from any meristematic part of a cotton plant can be used to produce a friable callus. Thus, the cells from the cotton explant can from cotton apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and/or cambial meristematic cells (CMC).

Preparing a friable callus may include contacting the cells of a cotton explant with a callus induction medium. The callus induction medium may facilitate the division of at least a subset of cells of a plant explant. Using the callus induction medium results in dedifferentiated cell masses. The cells in these masses can be subsequently cultured, which may include the use of a callus growth medium.

Certain aspects of the invention require the use of plant hormone(s) and/or growth regulator(s) (including auxins, gibberilins, etc.). The hormones/regulators can be used, for example, in the mediums described herein for culturing cotton cells. Plant hormones and/or growth regulators (including auxins, gibberilins, etc.) can be derived from naturally occurring sources, synthetically produced, or semi-synthetically produced, i.e. starting from naturally derived starting materials then synthetically modifying said materials. These modifications can be conducted using conventional methods as envisioned by a skilled worker. The following references include plant hormones and/or growth regulators (including auxins, gibberilins, etc.) for plant cell composition as described hereinbelow or described anywhere else herein: Gaspar et al. In Vitro Cell. Dev. Biol Plant, 32, 272-289, October-December 1996 and Zhang et al. Journal of Integrative Agriculture, 2017, 16(8): 1720-1729; the contents of each of which (particularly, all the plant hormones and/or plant growth regulators) are incorporated by reference herein. In particular, one of skill in the art will understand that certain gibberilins are capable of facilitating plant cell elongation.

In some aspects, plant hormones and/or growth regulators used in the present invention are exemplified by those in Table A.

TABLE A
Exemplary plant hormones or plant growth regulators
and exemplary applications in plant cell engineering.

| | | | | | | |
|---|---|---|---|---|---|---|
| indole butyric acid | IBA | Y | Y | Y | Y | |
| 2,4- | 2,4 D | Y | Y | Y | Y | |
| dichlorophenoxyacetic | | | | | | |
| acid | | | | | | |
| naphthaleneacetic acid | NAA | Y | Y | Y | Y | |
| para- | pCPA | Y | Y | Y | Y | |
| chlorophenoxyacetic | | | | | | |
| acid | | | | | | |
| β-naphthoxyacetic | NOA | Y | Y | Y | Y | |
| acid | | | | | | |
| 2-benzothiazole acetic | BTOA | Y | Y | Y | Y | |
| acid | | | | | | |
| picloram | PIC | Y | Y | Y | Y | |
| 2,4,5- | 2,4,5-T | Y | Y | Y | Y | |
| trichlorophenoxyacetic | | | | | | |
| acid | | | | | | |
| phenylacetic acid | PAA | Y | Y | Y | Y | |
| kinetin | KIN | Y | Y | Inhibitor | ND | |
| 6-benzylaminopurine | 6BA | Y | Y | Inhibitor | ND | |
| N6-(2-isopentenyl) | 2iP | Y | Y | Inhibitor | ND | |
| adenine | | | | | | |
| zeatin | ZEA | Y | Y | Inhibitor | ND | |
| gibberellin A1 | GA1 | ND | ND | Y | ND | Control fiber |
| gibberellic acid | GA3 | ND | ND | Y | ND | |
| gibberellin A4 | GA4 | ND | ND | Y | ND | strength, micronaire |
| gibberellin A7 | GA7 | ND | ND | Y | ND | and maturation |
| ethylene | — | ND | ND | Y | ND | |
| brassinolide | BR | ND | ND | Y | Y | |
| jasmonic acid | JA | ND | ND | Y | ND | |

“Y” indicates that the corresponding plant hormone or plant growth regulator in the row can be used for the application indicated in the column heading.
“Inhibitor” indicates that the corresponding plant hormone or plant growth regulator in the row can be used for inhibiting the activity indicated in the column heading.
“ND” indicates that effect(s) of the corresponding plant hormone or plant growth regulator for the application indicated in the column heading is not yet determined (at least to some extent).

In certain aspects, the invention uses an induction medium or callus induction medium. The callus induction medium described herein can be configured to facilitate division of at least a subset of cells of a plant explant. For example, the callus induction medium can facilitate or promote induction of a cotton plant callus. The callus induction medium can comprise a diluted basal medium (i.e., from 1:1.5 to 1:5, from 1:1.5 to 1:4, from 1:1.5 to 1:3, etc.). The callus induction medium can comprise one or more salts, macronutrients, micronutrients, organic molecules, and/or hormones (such as those that can facilitate or promote induction). The callus induction medium can be a liquid at about 25° C. Alternatively, the callus induction medium can be not a liquid at a specified temperature. In some embodiments, the callus induction medium is not a liquid at about 25° C. In some embodiments, the callus induction medium can be a semi-solid medium (such as gelled) at 25° C.

Non-limiting examples of a semi-solid medium include soft agar, soft agarose, soft methylcellulose, xantham gum, gellan gum, carrageenan, isabgol, guar gum, other soft polymeric gels, or any other gelling agent known in the art. The callus induction medium can comprise agar. In some embodiments, the callus induction medium can be agar-free. In some embodiments, the callus induction medium is free of any gelling agent. In some embodiments, the callus induction medium that is agar- or gelling agent-free can be a liquid. In some embodiments, the callus induction medium that is agar- or gelling agent-free can be a solid. In some embodiments, the callus induction medium that is agar-free can be a gel. In some embodiments, the callus induction medium that is agar-free can comprise an agarsubstitute. In some embodiments, the callus induction medium can have a pH. The pH of the callus induction medium can be appropriate for induction of a plant callus. In some embodiments, the pH of the callus induction medium can be optimized for induction of a plant callus. In some embodiments, the pH of the callus induction medium can be from 5.3 to 6.3. In some embodiments, the pH of the callus induction medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values.

The present disclosure includes callus mediums or callus growth mediums, and their use in the in vitro methods for producing cotton. The callus growth medium described herein can facilitate or promote growth of a plant callus and/or produce a proliferating cell aggregate. The callus growth medium can be a gel medium, and in some embodiments, can comprise agar and/or another gelling agent and a mixture of macronutrients and micronutrients for the plant type of the plant callus. In some cases, the callus medium can be enriched with nitrogen, phosphorus, or potassium. In some cases, a callus growth medium can be a liquid medium. In some embodiments, the callus growth medium can comprise at least one plant hormone or growth regulator (including auxins, gibberilins, etc.), or at least two plant hormones or growth regulators, or at least three plant hormones or growth regulators, or at least four plant hormones or growth regulators, or at least five plant hormones or growth regulators, or at least six plant hormones or growth regulators, or at least seven plant hormones or growth regulators, or at least eight plant hormones or growth regulators. The at least one plant hormone or plant growth regulator (or at least two, at least three, at least four, at least five, or at least six plant hormones or plant growth regulators) (including auxins, gibberilins, etc.) can be any one or combination selected from the group consisting of indole acetic acid (IAA), Indoyl-3-acrylic acid, 4-Cl-Indoyl-3-acetic acid, Indoyl-3-acetylaspartate, indole-3-acetaldehyde, indole-3-acetonitrile, indole-3-lactic acid, indole-3-propionic acid, indole-3-pyruvic acid, indole butyric acid (IB A), 2,4-dichlorophenoxyacetic acid (2,4 D), tryptophan, phenylacetic acid (PAA), Glucobrassicin, naphthaleneacetic acid (NAA), picloram (PIC), Dicamba, ethylene, para-chlorophenoxyacetic acid (pCPA), P-naphthoxyacetic acid (NOA), benzo(b)selenienyl-3 acetic acid, 2-benzothiazole acetic acid (BTOA), N6-(2-isopentenyl) adenine (2iP), zeatin (ZEA), dihydro-Zeatin, Zeatin riboside, kinetin (KIN), 6-(benzyladenine)-9-(2-tetrahydropyranyl)-9H-purine, 2, 4, 5, -trichlorophenoxyacetic acid (2,4,5-T), 6-benzylaminopurine (6BA), 1,3-diphenylurea, N-(2-chloro-4-pyridyl)-N′-phenylurea, (2, 6-di chi oro-4-pyridyl)-N′-phenylurea, N-phenyl-N′-1,2,3-thiadiazol-5-ylurea, gibberellin As, gibberellin Al (GAI), gibberellic acid (GA3), gibberellin A4 (GA4), gibberellin A7 (GA7), brassinolide (BR), jasmonic acid (J A), gibberellin As, gibberellin A32, gibberellin A9, 15-P—OH-gibberellin A3, 15-P—OH-gibberellin As, 12-P—OH-gibberellin As, 12-a-gibberellin As, salicylic acid, (−)jasmonic acid, (+)-7-isojasmonic acid, putrescine, spermidine, spermine, oligosaccharins, and stigmasterol. The at least one plant hormone or plant growth regulator (or at least two, at least three, at least four, at least five, or at least six plant hormones or plant growth regulators) (including auxins, gibberilins, etc.) can be any one or combination selected from the group consisting of indoyl-3-acetic acid, indoyl-3-acrylic acid, indoyl-3-butyric acid, 4-Cl-Indoyl-3-acetic acid, Indoyl-3-acetyl aspartate, indole-3-acetaldehyde, indole-3-acetonitrile, indole-3-lactic acid, indole-3-propionic acid, indole-3-pyruvic acid, tryptophan, phenylacetic acid, Glucobrassicin, 2,4-Dichloropheny oxyacetic acid, 1-naphthaleneacetic acid, Dicamba, Pichloram, ethylene, benzo(b)selenienyl-3 acetic acid, /ra/z.s-Zeatin, N⁶-(2-isopentyl)adenine, t/z/ryv/ra-Zeatin, Zeatin riboside, Kinetin, benzylamide, 6-(benzyladenine)-9-(2-tetrahydropyranyl)-9H-purine, 1,3-diphenylurea, N-(2-chloro-4-pyridyl)-N′-phenylurea, (2,6-dichloro-4-pyridyl)-N′-phenylurea, N-phenyl-N′-1,2,3-thiadiazol-5-ylurea, Gibberellin Ai, Gibberellin A3, Gibberellin A4, Gibberellin As, Gibberellin A7, Gibberellin As, Gibberellin A32, Gibberellin A9, 15-P—OH Gibberellin A3, 15-P—OH Gibberellin As, 12-P—OH Gibberellin As, 12-a-Gibberellin As, salicylic acid, jasmonic acid, (−) jasmonic acid, (+)-7-iso-jasmonicacid, putrescine, spermidine, spermine, oligosaccharins, brassinolide, and stigmasterol. The at least one plant hormone or plant growth regulator (or at least two, at least three, at least four, at least five, or at least six plant hormones or plant growth regulators) (including auxins, gibberilins, etc.) can be any one or combination selected from the group consisting of indole acetic acid (IAA), indole butyric acid (IBA), 2,4-dichlorophenoxyacetic acid (2,4 D), naphthaleneacetic acid (NAA), para-chlorophenoxyacetic acid (pCPA), P-naphthoxyacetic acid (NOA), 2-benzothiazole acetic acid (BTOA), picloram (PIC), 2, 4, 5, -trichlorophenoxyacetic acid (2,4,5-T), phenylacetic acid (PAA), kinetin (KIN), 6-benzylaminopurine (6BA), N6-(2-isopentenyl) adenine (2iP), zeatin (ZEA), gibberellin Al (GAI), gibberellic acid (GA3), gibberellin A4 (GA4), gibberellin A7 (GA7), ethylene, brassinolide (BR), and jasmonic acid (JA).

In certain aspects, the callus growth medium can be a liquid at about 25° C. In some embodiments, the callus growth medium can be not a liquid at about 25° C. In some embodiments, the callus growth medium can be a semi-solid medium (such as gelled) at 25° C. Non-limiting examples of a semi-solid medium include soft agar, soft agarose, soft methylcellulose, xantham gum, gellan gum, carrageenan, isabgol, guar gum, other soft polymeric gels, or any other gelling agent known in the art. In some embodiments, the callus growth medium can comprise agar. In some embodiments, the callus growth medium can be agar-free. In some embodiments, the callus growth medium is free of any gelling agent. In some embodiments, the callus growth medium that is agar- or gelling agent-free can be a liquid. In some embodiments, the callus growth medium that is agar- or gelling agent-free can be a solid. In some embodiments, the callus growth medium that is agar-free can be a gel. In some embodiments, the callus growth medium that is agar-free can comprise an agarsubstitute.

In some embodiments, the callus growth medium can have a pH. The pH of the callus growth medium can be appropriate for growing a plant callus and/or producing a proliferating cell aggregate. In some embodiments, the pH of the callus growth medium can be optimized for growing a plant callus and/or producing a proliferating cell aggregate. In some embodiments, the pH of the callus growth medium can be from 5.3 to 6.3. In some embodiments, the pH of the callus growth medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values.

The present invention includes cell culture mediums (e.g., a multiplication/duplication mediums), and their use in the in vitro methods for producing cotton described herein. In some embodiments, the cell culture medium described herein can facilitate or promote proliferation of a cell population, or a proliferating cell aggregate. The cell culture medium can comprise one or more salts, macronutrients, micronutrients, organic molecules, and/or hormones (such as those that can facilitate or promote proliferation). In some cases, the cell culture medium can be configured to proliferate a cell population, such as a proliferating cell aggregate. The cell culture medium can comprise an enzyme that can degrade a plant cell wall of a plant cell of a cell population, or a proliferating cell aggregate. In some embodiments, the enzyme can be a pectocellulolytic enzyme. In some embodiments, the enzyme can comprise cellulase, hemicellulose, cellulysin, or a combination thereof. In some embodiments, the cell culture medium can have a pH. The pH of the cell culture medium can be appropriate for culturing a cell population, or a proliferating cell aggregate.

In some embodiments, the pH of the cell culture medium can be optimized for culturing a cell population, such as a proliferating cell aggregate. In some embodiments, the pH of the cell culture medium can be optimized for cell division. In some embodiments, the pH of the cell culture medium can be from 5.3 to 6.3. In some embodiments, the pH of the cell culture medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values. In some embodiments, the cell culture medium can have a different pH than a callus growth medium. In some embodiments, the cell culture medium can have a same pH as a callus growth medium. In some embodiments, the pH of the cell culture medium can differ from a pH of a callus growth medium by less than 0.1, less than 0.2, or less than 0.3 units. For example, the pH of a cell culture medium can differ from a pH of a callus growth medium by less than 0.2 units.

Preferably, a cell culture medium of the present disclosure includes one or more of MS, B5, glucose, sucrose, Kinetin, 2,4-dichlorophenoxyacetic acid (2,4-D), NAA, and coconut water. Preferably, the cell culture medium comprises 2,4-D. In certain aspects, the cell culture medium includes MS, B5, glucose/sucrose, and 2,4-D.

The present invention also includes recovery mediums, and their use in the in vitro methods for producing cotton fiber. A recovery medium can be used, for example, for recovery of cotton cell inoculum after cryopreservation. Some embodiments described herein are related to a recovery medium. In some embodiments, the recovery medium described herein can be a medium that can facilitate or promote recovery of cotton cells. The recovery medium can comprise one or more salts, macronutrients, micronutrients, organic molecules, and/or hormones that can facilitate or promote elongation.

The present invention includes elongation mediums, and their use in the in vitro methods for producing cotton fiber. The elongation mediums described herein can facilitate or promote elongation of cells capable of being elongated, for example, elongation of cotton cells. The elongation mediums described herein can comprise one or more salts, macronutrients, micronutrients, organic molecules, and/or hormones (such as those that can facilitate or promote elongation). In some embodiments, the elongation mediums can be configured to facilitate a release of a phenolic compound from a vacuole from a cotton cell. In some embodiments, the phenolic compound (such as O-diphenol) is configured to initiate fiber differentiation by inhibiting indoleacetic acid (IAA) oxidase and/or increase an intracellular auxin level. In some embodiments, the elongation medium can comprise at least one plant hormone or growth regulator (including auxins, gibberilins, etc.), or at least two plant hormones or growth regulators, or at least three plant hormones or growth regulators, or at least four plant hormones or growth regulators, or at least five plant hormones or growth regulators, or at least six plant hormones or growth regulators, or at least seven plant hormones or growth regulators, or at least eight plant hormones or growth regulators. The at least one plant hormone or plant growth regulator (or at least two, at least three, at least four, at least five, or at least six plant hormones or plant growth regulators) (including auxins, gibberilins, etc.) can be any one or combination selected from the group consisting of indole acetic acid (IAA), Indoyl-3-acrylic acid, 4-Cl-Indoyl-3-acetic acid, Indoyl-3-acetylaspartate, indole-3-acetaldehyde, indole-3-acetonitrile, indole-3-lactic acid, indole-3-propionic acid, indole-3-pyruvic acid, indole butyric acid (IB A), 2,4-dichlorophenoxyacetic acid (2,4 D), tryptophan, phenylacetic acid (PAA), Glucobrassicin, naphthaleneacetic acid (NAA), picloram (PIC), Dicamba, ethylene, parachlorophenoxyacetic acid (pCPA), P-naphthoxyacetic acid (NOA), benzo(b)selenienyl-3 acetic acid, 2-benzothiazole acetic acid (BTOA), N6-(2-isopentenyl) adenine (2iP), zeatin (ZEA), t/z/ryv/ra-Zeatin, Zeatin riboside, kinetin (KIN), 6-(benzyladenine)-9-(2-tetrahydropyranyl)-9H-purine, 2,4,5,-trichlorophenoxyacetic acid (2,4,5-T), 6-benzylaminopurine (6BA), 1,3-diphenylurea, N-(2-chloro-4-pyridyl)-N′-phenylurea, (2, 6-di chi oro-4-pyridyl)-N′-phenylurea, N-phenyl-N′-1,2,3-thiadiazol-5-ylurea, gibberellin As, gibberellin Al (GAI), gibberellic acid (GA3), gibberellin A4 (GA4), gibberellin A7 (GA7), brassinolide (BR), jasmonic acid (J A), gibberellin As, gibberellin A32, gibberellin A9, 15-P—OH-gibberellin A3, 15-P—OH-gibberellin A5, 12-P—OH-gibberellin As, 12-a-gibberellin As, salicylic acid, (−)jasmonic acid, (+)-7-isojasmonic acid, putrescine, spermidine, spermine, oligosaccharins, and stigmasterol. The at least one plant hormone or plant growth regulator (or at least two, at least three, at least four, at least five, or at least six plant hormones or plant growth regulators) (including auxins, gibberilins, etc.) can be any one or combination selected from the group consisting of indoyl-3-acetic acid, indoyl-3-acrylic acid, indoyl-3-butyric acid, 4-Cl-Indoyl-3-acetic acid, Indoyl-3-acetyl aspartate, indole-3-acetaldehyde, indole-3-acetonitrile, indole-3-lactic acid, indole-3-propionic acid, indole-3-pyruvic acid, tryptophan, phenylacetic acid, Glucobrassicin, 2,4-Dichlorophenyoxyacetic acid, 1-naphthaleneacetic acid, Dicamba, Pichloram, ethylene, benzo(b)selenienyl-3 acetic acid, /ra/z.s-Zeatin, N⁶-(2-isopentyl)adenine, t/z/rjv/zYz-Zeatin, Zeatin riboside, Kinetin, benzylamide, 6-(benzyladenine)-9-(2-tetrahydropyranyl)-9H-purine, 1,3-diphenylurea, N-(2-chloro-4-pyridyl)-N′-phenylurea, (2,6-dichloro-4-pyridyl)-N′-phenylurea, N-phenyl-N′-1,2,3-thiadiazol-5-ylurea, Gibberellin Ai, Gibberellin A3, Gibberellin A4, Gibberellin As, Gibberellin A7, Gibberellin As, Gibberellin A32, Gibberellin A9, 15-P—OH-Gibberellin A3, 15-P—OH-Gibberellin As, 12-P—OH-Gibberellin As, 12-a-Gibberellin As, salicylic acid, jasmonic acid, (−) jasmonic acid, (+)-7-iso-jasmonicacid, putrescine, spermidine, spermine, oligosaccharins, brassinolide, and stigmasterol. The at least one plant hormone or plant growth regulator (or at least two, at least three, at least four, at least five, or at least six plant hormones or plant growth regulators) (including auxins, gibberilins, etc.) can be any one or combination selected from the group consisting of indole acetic acid (IAA), indole butyric acid (IBA), 2,4-dichlorophenoxyacetic acid (2,4 D), naphthaleneacetic acid (NAA), para-chlorophenoxyacetic acid (pCPA), P-naphthoxyacetic acid (NOA), 2-benzothiazole acetic acid (BTOA), picloram (PIC), 2, 4, 5, -trichlorophenoxyacetic acid (2,4,5-T), phenylacetic acid (PAA), kinetin (KIN), 6-benzylaminopurine (6BA), N6-(2-isopentenyl) adenine (2iP), zeatin (ZEA), gibberellin Al (GAI), gibberellic acid (GA3), gibberellin A4 (GA4), gibberellin A7 (GA7), ethylene, brassinolide (BR), and jasmonic acid (J A).

In certain aspects, the callus growth medium can be a liquid at about 25° C. In some embodiments, the callus growth medium can be not a liquid at about 25° C. In some embodiments, the callus growth medium can be a semi-solid medium (such as gelled) at 25° C. The present Inventors discovered that a semi-solid medium provides better results than a liquid medium. Non-limiting examples of a semi-solid medium include soft agar, soft agarose, soft methylcellulose, xantham gum, gellan gum, carrageenan, isabgol, guar gum, other soft polymeric gels, or any other gelling agent known in the art. In some embodiments, the callus growth medium can comprise agar. In some embodiments, the callus growth medium can be agar-free. In some embodiments, the callus growth medium is free of any gelling agent. In some embodiments, the callus growth medium that is agar- or gelling agent-free can be a liquid. In some embodiments, the callus growth medium that is agar- or gelling agent-free can be a solid. In some embodiments, the callus growth medium that is agar-free can be a gel. In some embodiments, the callus growth medium that is agar-free can comprise an agar-substitute.

In some embodiments, the elongation medium can have a pH. The pH of the elongation medium can be appropriate for producing/inducing an elongated cell, such as an elongated cotton cell or a plurality of elongated cotton cells. In some embodiments, the pH of the elongation medium can be optimized for cell elongation (such as cotton cell elongation). In some embodiments, the pH of the elongation medium can be from 5.3 to 6.3. In some embodiments, the pH of the elongation medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values.

The present invention includes elongation mediums, and their use in the in vitro methods for producing cotton fiber. In some embodiments, the maturation mediums described herein can facilitate or promote maturation of cells, such as maturation of cotton cells. A maturation medium can comprise one or more salts, macronutrients, micronutrients, organic molecules, and/or hormones (such as those that can facilitate or promote maturation). In some embodiments, the maturation medium can comprise a maturation reagent. In some embodiments, the maturation reagent of the maturation medium can be a wall-regeneration reagent.

The present invention includes the use of proliferating cell aggregates, and their creation, for use in the in vitro methods of cotton fiber production. In some embodiments, the plant cell composition as described hereinbelow or described anywhere else herein can be derived from the proliferating cell aggregate. The proliferating cell aggregate can be an aggregate of plant cells that are proliferating. Proliferating cells in an aggregate can be attached or connected to each other, for example, via cell-to-cell interactions. The proliferating cell aggregate can be a friable callus is friable, which is not sticky or soft, but is also not so hard or dense that it cannot be physically broken or crumbled. A friable callus thus differs “a hard callus”, which is compact and brittle, and thus not amenable to being broken or crumbled. Preferably the callus is a friable callus. The present Inventors discovered that a friable callus can have individual cells dissociated from the callus using simple mechanical manipulation.

Proliferating cells can be of one type (a homogenous aggregate) or of two or more types (a heterogeneous aggregate). The proliferating cell aggregate can be a mixed aggregate (e.g., where cell types are mixed together), a clustering aggregate (e.g., where cells of different types are tending toward different parts of the aggregate), or a separating aggregate (where cells of different types are pulling apart from each other). Cells of the proliferating cell aggregate can divide at a rate greater than a cell division rate of remaining cells in said plant callus. In some embodiments, cells of the proliferating cell aggregate can divide at a rate that can be at least 2, 3, 4, 5, 6, 7, 8, 9, 10, 20, 30, 40, or 50 times greater than a cell division rate of plant callus cells.

The present invention includes the use of cells from a cotton plant cell callus and methods for preparing such a callus. The plant callus can be a growing mass of plant parenchyma cells. However, the Inventors discovered that, surprisingly, cells from any meristematic part of a cotton plant are sufficient for callus induction. Thus, the plant callus can be created using cells obtained or derived from cotton apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and/or cambial meristematic cells (CMC). In some cases, the mass of plant parenchyma cells can be unorganized. The plant callus can be collected from cells covering the wound of a plant or plant part. Preferably, the plant callus is created by inducing a plant tissue sample (e.g., an explant) with a callus induction medium. In some cases, induction of an explant can occur after surface sterilization and plating onto a medium in vitro (e.g., in a closed culture vessel such as a Petri dish). Induction can comprise supplementing the medium with plant growth regulators, such as auxins, cytokinins, or gibberellins to initiate callus formation. Induction can be performed at a temperature of, or of about, 20° C., 25° C., 28° C., 30° C., 35° C., or 40° C., or a range between any two foregoing values.

Compositions comprising cotton plant cells are included in the present invention. The plant cell compositions described herein can be a final product of a method for preparation of cell bank stocks provided herein. The plant cell compositions can be compositions of engineered cells, or a compositions of wildtype cells. The plant cell compositions can be cell bank stocks. The plant cell compositions can comprise a plurality of plant cells obtained by growing a callus in a growth medium to produce a proliferating cell aggregate followed by culturing the proliferating cell aggregate.

The plant cell compositions described herein can be in a growth phase. The growth phase can comprise cell division, cell enlargement, and/or cell differentiation. The growth phase comprising cell division can be an exponential growth phase (e.g., dowaiting). In some embodiments, the exponential growth phase can occur as cells are mitotic. In some embodiments, during exponential growth, each generation of cells can be twice as numerous as the previous generation. In some embodiments, not all cells may survive in a given generation. In some embodiments, each generation of cells can be less than twice as numerous as the previous generation. In some embodiments, the exponential growth phase can be determined (e.g., quantified or identified) by a cell viability assay. In some embodiments, another aspect of the plant cell composition can be determined by a cell viability assay. In some embodiments, the cell viability assay can be an assay that can determine the ability of a cell to maintain or recover viability. In some embodiments, the cells of the plant cell composition can be assayed for their ability to divide or for active cell division. In some embodiments, the cell viability assay can be an ATP test, calcein AM, clonogenic assay, ethidium homodimer assay, Evans blue, fluorescein diacetate hydrolysis/propidium iodide staining (FDA/PI staining), flow cytometry, formazan-based assays (e.g., MTT or XTT), green fluorescent protein based assays, lactate dehydrogenase (LDH) based assays, methyl violet, neutral red uptake, propidium iodide, resazurin, trypan blue, or a TUNEL assay. In some embodiments, the cell viability assay can determine a cytoplasmic level of diphenol compounds in the plant cell composition.

Also provided herein are bioreactors configured to produce any one or more compositions associated with the in vitro production of fiber as disclosed herein.

In some embodiments, a bioreactor can be configured to produce a cell bank stock. In some embodiments, a bioreactor can be configured to carry out a method for preparing a cell bank stock. In some such cases, a bioreactor can be configured to utilize components of a kit for preparation of a cell bank stock, such as a callus growth medium and/or a multiplication medium.

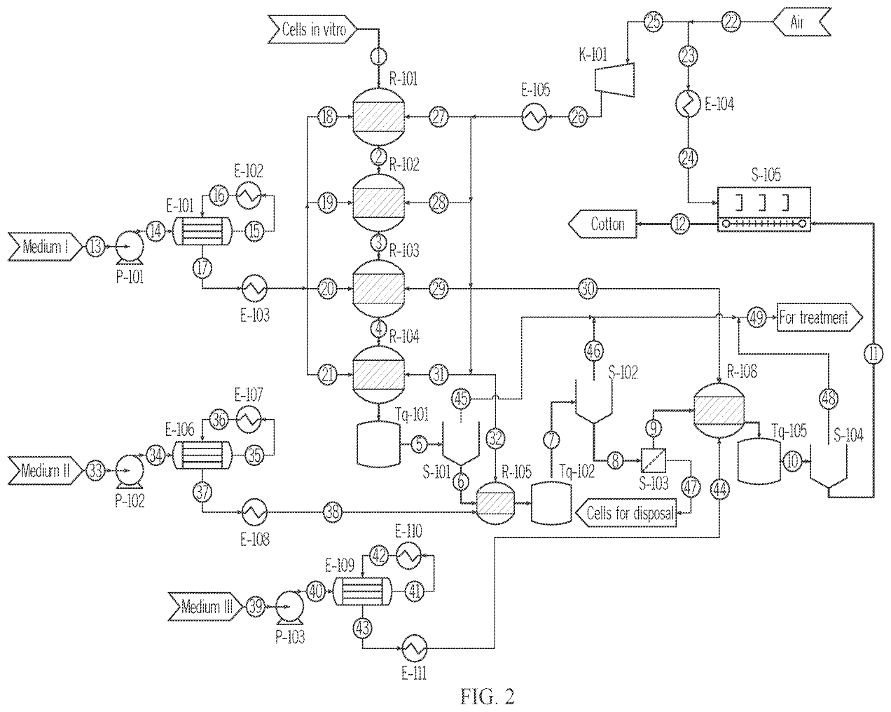

FIG. 2 provides a flow chart illustrating an example of different processes that can be performed by a bioreactor, and how these processes can be interconnected.

In some embodiments, a bioreactor can be configured to produce a cotton fiber. In some embodiments, a bioreactor can be configured to carry out a method for large scale cotton fiber production. In some embodiments, a bioreactor can be configured to carry out a method for rapid cotton fiber production. In some embodiments, a bioreactor can be configured to utilize components of a kit for large scale fiber production. In some embodiments, a bioreactor can be configured to utilize components of a kit for rapid fiber production.

In some embodiments, a bioreactor can be configured to produce engineered cotton. In some embodiments, a bioreactor can be configured to utilize components of a kit for production of engineered cotton, which can comprise elements of kits provided herein.

The present invention includes computer systems that are programmed to implement methods of the disclosure.

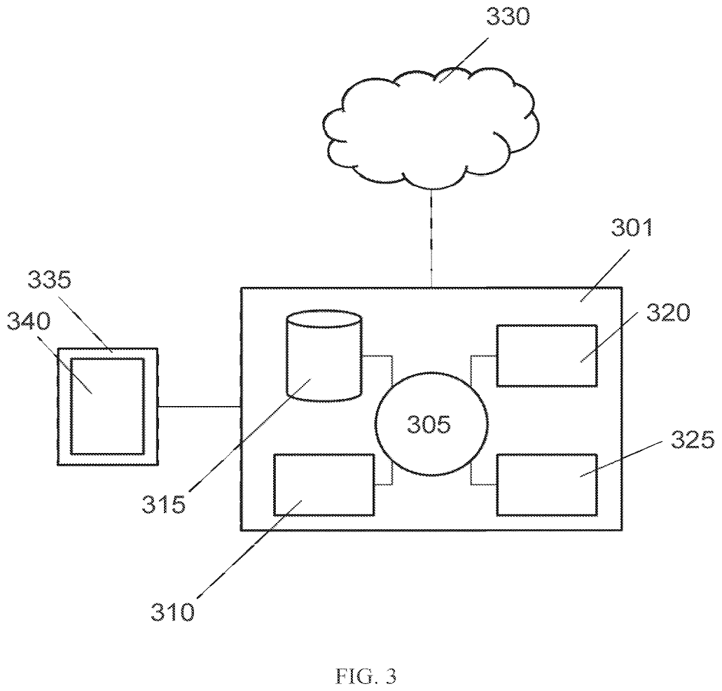

FIG. 3 shows a computer system 301 that is programmed or otherwise configured to provide and/or implement instructions for or means of implementation of induction, callus growth, cell culture, elongation, or maturation. The computer system 301 can regulate various aspects of induction, callus growth, cell culture, elongation, or maturation of the present disclosure. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 201 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine-readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, instructions for or means of implementation of induction, callus growth, cell culture, elongation, or maturation. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, provide and/or execute instructions for or means of implementation of induction, callus growth, cell culture, elongation, or maturation.

The present invention is further described by the following non-limiting Examples.

The cotton cells may be modified to express, or selected for expression of, at least one selected gene.

The selected gene may include a gene selected for improvement and/or modulation of cotton fiber development in vitro. These genes may improve/modulate cotton fiber development in cotton cells and/or cotton plants. However, genes that do not modulate/improve cotton fiber development in plants may nevertheless modulate/improve cotton fiber development using the in vitro methods disclosed herein. The gene selected for improvement and/or modulation of cotton fiber development may be endogenous to one or more species or varietal of cotton plant, may be a gene from, or derived from, another species, or may be a synthetic gene. The gene may be, or derived from, a cotton fiber development gene. Expression or overexpression of genes such as GhHOX3, GhHD-1, SPL5, GaMYB2, iaaM, GhPIN3a, GhWlim5. and GhFP1 has been shown to have a positive impact on cotton fiber growth (Cai, C., et al., 2018; Liu, Z. et al., 2020; Shan, C. et al., 2014; Walford, S. et al., 2012; Wang, S. et al., 2004; Zhang, M., et al., 2011; Mei, G. et al., 2019; Iqbal, A. et al., 2020).

In some embodiments, the cotton (or engineered cotton) described herein can be derived from a Gossypium species. The Gossypium species can be selected from the group consisting of G. arbor eum. G. anomalum. G. armouriamim. G. klolzchianum. and G. raimondii. The cotton (or engineered cotton) can be derived from a Gossypium species selected from the group consisting of G. hirsiilum. G. arbor eum, G. barbadense, G. anomalum, G. armourianum, G. klolzchianum, and G. raimondii. The cotton (or engineered cotton) can be Gossypium hirsiiliim. Gossypium bar bade use, Gossypium arboretum, Gossypium herbaceum, or another species of cotton.

The cotton cells used in the methods for producing cotton fiber in vitro may include the use of cotton cells that have a differently expressed gene (DEG). Cotton cells of the present disclosure can be subject to a mutagenic process to give rise the DEG. This process can occur in vitro and without ever growing a whole cotton plant with the DEG.

Mutagenesis can be achieved by raditiation and/or chemical means, including EMS or sodium azide treatment of seed, or gamma irradiation. Chemical mutagenesis favors nucleotide substitutions rather than deletions. Heavy ion beam (HIB) irradiation is a known technique for mutagenesis. Ion beam irradiation has two physical factors, the dose (gy) and LET (linear energy transfer, keV/um) for biological effects that determine the level of DNA damage and the size of any DNA deletion(s), and these can be adjusted according to change the extent of mutagenesis.

Biological agents can also be used to create site-specific mutations in cotton cells. These agents may include enzymes that cause double stranded breaks in DNA, which stimulate endogenous repair mechanisms. These enzymes include endonucleases, zinc finger nucleases, transposases and site-specific recombinases.

In certain aspects, the cotton cells may be transgenic. A transgenic cell is a genetically modified plant cell in which an endogenous genome or gene is supplemented or modified by an introduced foreign or exogenous gene or sequence. Often, these genes are under the control of a promoter to which they are operably connected. A transgene is a foreign exogenous gene or sequence introduced into the plant.

Also provided herein are bioreactors configured to produce any one or more compositions associated with the in vitro production of fiber as disclosed herein

In some embodiments, a bioreactor can be configured to produce a cell bank stock. In some embodiments, a bioreactor can be configured to carry out a method for preparing a cell bank stock. In some such cases, a bioreactor can be configured to utilize components of a kit for preparation of a cell bank stock, such as a callus growth medium and/or a multiplication medium.

Disclosed herein, in some embodiments, are cotton (or engineered cotton), cotton fibers (or engineered cotton fibers), compositions comprising cotton (or engineered cotton), and compositions comprising cotton fibers (or engineered cotton fibers) produced using the presently disclosed in vitro methods. In certain aspects, the methods produce cotton (or engineered cotton), as described hereinbelow.

In certain aspects, methods of the invention produce cotton (or engineered cotton) described herein having a dry mass of at least 10 grams per liter (g/L) fresh weight (FW) (e.g., grams of dry mass obtained per liter of fresh weight cotton cells). In some embodiments, the dry mass of the cotton (or engineered cotton) can be at least 50 grams per liter (g/L) fresh weight (FW). In some embodiments, the dry mass of the cotton (or engineered cotton) can be at least 100 grams per liter (g/L) fresh weight (FW). In some embodiments, the dry mass of the cotton (or engineered cotton) can be from 50 grams per liter (g/L) fresh weight (FW) to 500 g/L (FW). In some embodiments, the dry mass of the cotton (or engineered cotton) can be from 100 grams per liter (g/L) fresh weight (FW) to 500 g/L (FW). In some embodiments, the dry mass of the cotton (or engineered cotton) can be from 100 grams per liter (g/L) fresh weight (FW) to 300 g/L (FW). In some embodiments, the dry mass of the cotton (or engineered cotton) can have a dry mass of about 50 grams per liter (g/L) fresh weight (FW), about 100 g/L FW, about 200 g/L FW, about 300 g/L FW, about 400 g/L FW, about 500 g/L FW, about 600 g/L FW, about 700 g/L FW, about 800 g/L FW, about 900 g/L FW, or about 1000 g/L FW, or a range between any of the foregoing values. In some embodiments, the dry mass of the cotton (or engineered cotton) can have a dry mass of at least 50 grams per liter (g/L) fresh weight (FW), at least 100 g/L FW, at least 200 g/L FW, at least 300 g/L FW, at least 400 g/L FW, at least 500 g/L FW, at least 600 g/L FW, at least 700 g/L FW, at least 800 g/L FW, at least 900 g/L FW, or at least 1000 g/L FW, or a range between any of the foregoing values. In some embodiments, the cotton (or engineered cotton) described herein can have a dry mass of at least 50 milligrams (mg). In some embodiments, the cotton can have a dry mass of at least 10 mg, at least 20 mg, at least 30 mg, at least 40 mg, at least 50 mg, at least 60 mg, at least 70 mg, at least 80 mg, at least 90 mg, at least 100 mg, at least 200 mg, at least 300 mg, at least 400 mg, at least 500 mg, or at least 1000 mg. In some embodiments, the cotton can have a dry mass of at least 1 gram (g), at least 5 g, at least 10 g, at least 50 g, at least 100 g, at least 500 g, at least 1 kg, at least 5 kg, at least 10 kg, at least 50 kg, or at least 100 kg.

In certain aspects, methods of the invention produce cotton (or engineered cotton) described herein having about, 0.1%, 0.2%, 0.3%, 0.4%, 0.5%, 0.6%, 0.7%, 0.8%, 0.9%, 1%, 1.5%, 2%, 2.5%, or 3%, or a range between any two foregoing, by dry weight of a trash content (TC). In some embodiments, the cotton (or engineered cotton) described herein can comprise at most, or comprise at most about, 0.1%, 0.2%, 0.3%, 0.4%, 0.5%, 0.6%, 0.7%, 0.8%, 0.9%, 1%, 1.5%, 2%, 2.5%, or 3% by dry weight of a trash content. In some embodiments, the cotton (or engineered cotton) described herein can comprise at least, or comprise at least about, 0.1%, 0.2%, 0.3%, 0.4%, 0.5%, 0.6%, 0.7%, 0.8%, 0.9%, 1, 1.5%, 2%, 2.5%, or 3% by dry weight of a trash content. In some embodiments, the cotton (or engineered cotton) can comprise at most 3% by dry weight of a trash content. In some embodiments, the cotton (or engineered cotton) can comprise at most 2% by dry weight of a trash content. In some embodiments, the cotton can comprise at most 1% by dry weight of a trash content. In some embodiments, the cotton can comprise at most 0.5% by dry weight of a trash content. In some embodiments, the cotton can comprise at most 0.2% by dry weight of a trash content. In some embodiments, the cotton can comprise at most 0.1% by dry weight of a trash content. In some embodiments, the trash content can be a non-lint substance (such as non-cotton substance and cottons with convolutions, strings, conjoint defects, motes, or broken seeds). The “trash content” of a cotton sample can be measured by a Premier G-Trash Tester.

In some embodiments, the cotton (or engineered cotton) described herein comprises cotton fibers. A cotton fiber of the cotton (or engineered cotton) can be an elongated cotton cell. In some embodiments, the cotton (or engineered cotton) described herein can comprise at least 95%, or at least 99% by dry weight cotton fibers. In some embodiments, the cotton (or engineered cotton) can comprise at least 99% by dry weight cotton fibers.

In some embodiments, the cotton fibers of the cotton (or engineered cotton) as described herein can comprise, by dry weight, a maximum threshold of a short fiber content (SFC). In some embodiments, the cotton fibers of the cotton (or engineered cotton) as described herein can comprise, or comprise about, 1%, 5%, 10%, 15%, 20%, 25%, or 30%, or a range between any two forgoing, by dry weight, a short fiber content (SFC). In some embodiments, the cotton fibers of the cotton (or engineered cotton) as described herein can comprise at most 30%, at most 25%, at most 20%, at most 15%, at most 10%, at most 5%, or at most 1%, by dry weight, a short fiber content (SFC). In some embodiments, the cotton fibers of the cotton (or engineered cotton) as described herein can comprise at most 10% by dry weight a short fiber content (SFC). In some embodiments, the cotton fibers of the cotton (or engineered cotton) as described herein can comprise at least 1%, at least 5%, at least 10%, at least 15%, at least 20%, at least 25%, or at least 30% by dry weight a short fiber content (SFC). In some embodiments, cotton fibers of the short fiber contents have a length no more than a pre-determined length (such as 0.5 inch, or any length from 2.2 to 3.0 centimeter (cm)).

In some embodiments, the cotton fibers of the cotton (or engineered cotton) as described herein (or the plurality of elongated cotton cells as obtained using a described anywhere else) can have an average fiber length of, or of about, 0.5, 0.6, 0.7, 0.8, 0.9, 1.0,

1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 centimeters (cm), or a range between any two foregoing values. In some embodiments, the cotton fibers of the cotton (or engineered cotton) (or the plurality of elongated cotton cells as obtained using the presently disclosed methods) have an average fiber length of from 1.1 centimeter (cm) to 4.0 cm. In some embodiments, the cotton fibers of the cotton (or engineered cotton) as described herein can have a length uniformity. The length uniformity can be an indicator of how similar the lengths of cotton fibers are in a cotton composition. In some embodiments, the cotton fibers can have a length uniformity of, or of about, 60%, 65%, 70%, 75%, 80%, 85%, 90%, 95%, or 99%, or a range between any two foregoing values. In some embodiments, the cotton fibers can have a length uniformity of at least 70%. In some embodiments, the cotton fibers can have a length uniformity of at least 50%, at least 55%, at least 60%, at least 65%, at least 70%, at least 75%, at least 80%, at least 85%, or at least 90%. In some embodiments, the cotton fibers can have a length uniformity of at most 50%, at most 55%, at most 60%, at most 65%, at most 70%, at most 75%, at most 80%, at most 85%, or at most 90%.

In some embodiments, the cotton fibers of the cotton (or engineered cotton) as described herein can comprise a secondary wall. The secondary wall of the cotton fibers described herein can have an average thickness. The average thickness of the secondary wall of the cotton fibers can be of, or of about, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5, 4.6, 4.7, 4.8, 4.9, 5.0, 5.5, or 6.0 micron (pm), or a range between any two foregoing values. The average thickness of the secondary wall of the cotton fibers can be at most, or at most about, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5, 4.6, 4.7, 4.8, 4.9, 5.0, 5.5, or 6.0 micron (pm).

The average thickness of the secondary wall of the cotton fibers can be at least, or at least about, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5, 4.6, 4.7, 4.8, 4.9, 5.0, 5.5, or 6.0 micron (pm). In some embodiments, the cotton fibers described herein can have an average thickness of a secondary wall of at least 4 pm.

In some embodiments, methods of the invention produce cotton (or engineered cotton) comprising a threshold amount of cellulose by dry weight. In some embodiments, the cotton fibers can comprise, or comprise about, by dry weight, 80%, 81%, 82%, 83%, 84%, 85%, 86%, 87%, 88%, 89%, 90%, 91%, 92%, 93%, 94%, 95%, 96%, 97%, 98%, or 99% cellulose, or a range between any two foregoing values. In some embodiments, the cotton fibers can comprise at least, or comprise at least about, by dry weight, 80%, 81%, 82%, 83%, 84%, 85%, 86%, 87%, 88%, 89%, 90%, 91%, 92%, 93%, 94%, 95%, 96%, 97%, 98%, or 99% cellulose. In some embodiments, the cotton fibers can comprise at most, or comprise at most about, by dry weight, 80%, 81%, 82%, 83%, 84%, 85%, 86%, 87%, 88%, 89%, 90%, 91%, 92%, 93%, 94%, 95%, 96%, 97%, 98%, or 99% cellulose. In some embodiments, the cotton fibers can comprise, by dry weight, from 88% to 96% cellulose. In some embodiments, the cotton fibers of the cotton (or engineered cotton) as described herein can comprise a threshold amount of protein by dry weight. In some embodiments, the cotton fibers can comprise, or comprise about, by dry weight, 0.7%, 0.8%, 0.9%, 1.0%, 1.2%, 1.3%, 1.4%, 1.5%, 1.6%, 1.7%, 1.8%, 1.9%, 2.0%, 2.1%, 2.2%, 2.3%, 2.4%, 2.5% protein, or a range between any two foregoing values. In some embodiments, the cotton fibers can comprise at least, or comprise at least about, by dry weight, 0.7%, 0.8%, 0.9%, 1.0%, 1.2%, 1.3%, 1.4%, 1.5%, 1.6%, 1.7%, 1.8%, 1.9%, 2.0%, 2.1%, 2.2%, 2.3%, 2.4%, 2.5% protein. In some embodiments, the cotton fibers can comprise at most, or comprise at most about, by dry weight, 0.7%, 0.8%, 0.9%, 1.0%, 1.2%, 1.3%, 1.4%, 1.5%, 1.6%, 1.7%, 1.8%, 1.9%, 2.0%, 2.1%, 2.2%, 2.3%, 2.4%, 2.5% protein. In some embodiments, the cotton fiber can comprise, by dry weight, from 1.1% to 1.9% protein.

In some embodiments, the cotton fibers of the cotton (or engineered cotton) as described herein can comprise a threshold amount of pectic substance by dry weight. In some embodiments, the cotton fibers can comprise, or comprise about, by dry weight, 0.5%, 0.6%, 0.7%, 0.8%, 0.9%, 1.0%, 1.1%, 1.2%, 1.3%, 1.4%, 1.5%, 1.6%, 1.7%, or 1.8% pectic substance, or a range between any two foregoing values. In some embodiments, the cotton fibers can comprise at least, or comprise at least about, by dry weight, 0.5%, 0.6%, 0.7%, 0.8%, 0.9%, 1.0%, 1.1%, 1.2%, 1.3%, 1.4%, 1.5%, 1.6%, 1.7%, or 1.8% pectic substance. In some embodiments, the cotton fibers can comprise at most, or comprise at most about, by dry weight, 0.5%, 0.6%, 0.7%, 0.8%, 0.9%, 1.0%, 1.1%, 1.2%, 1.3%, 1.4%, 1.5%, 1.6%, 1.7%, or 1.8% pectic substance. In some embodiments, the cotton fiber can comprise, by dry weight, from 0.7% to 1.2% pectic substance.

In some embodiments, the cotton fibers of the cotton (or engineered cotton) as described herein can comprise a threshold amount of ash by dry weight. In some embodiments, the cotton fibers can comprise, or comprise about, by dry weight, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0% ash, or a range between any two foregoing values. In some embodiments, the cotton fibers can comprise at least, or comprise at least about, by dry weight, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0% ash. In some embodiments, the cotton fibers can comprise at most, or comprise at most about, by dry weight, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0% ash. In some embodiments, the cotton fibers can comprise, by dry weight, from 0.7% to 1.6% ash.

In some embodiments, the cotton fibers of the cotton (or engineered cotton) as described herein can comprise a threshold amount of wax by dry weight. In some embodiments, the cotton fibers can comprise, or comprise about, by dry weight, 0.1%, 0.2%, 0.3%, 0.4%, 0.5%, 0.6%, 0.7%, 0.8%, 0.9%, 1.0%, 1.1%, 1.2%, 1.3%, 1.4%, 1.5% wax, or a range between any two foregoing values. In some embodiments, the cotton fibers can comprise at least, or comprise at least about, by dry weight, 0.1%, 0.2%, 0.3%, 0.4%, 0.5%, 0.6%, 0.7%, 0.8%, 0.9%, 1.0%, 1.1%, 1.2%, 1.3%, 1.4%, 1.5% wax. In some embodiments, the cotton fibers can comprise at most, or comprise at most about, by dry weight, 0.1%, 0.2%, 0.3%, 0.4%, 0.5%, 0.6%, 0.7%, 0.8%, 0.9%, 1.0%, 1.1%, 1.2%, 1.3%, 1.4%, 1.5% wax. In some embodiments, the cotton fibers can comprise, by dry weight, from 0.4% to 1.1% wax.

In some embodiments, the cotton fibers of the cotton (or engineered cotton) as described herein can comprise a threshold amount of sugar by dry weight. In some embodiments, the cotton fibers can comprise, or comprise about, by dry weight, 0.1%, 0.2%, 0.3%, 0.4%, 0.5%, 0.6%, 0.7%, 0.8%, 0.9%, 1.0%, 1.1%, 1.2%, 1.3%, 1.4%, 1.5% sugar, or a range between any two foregoing values. In some embodiments, the cotton fibers can comprise at least, or comprise at least about, by dry weight, 0.1%, 0.2%, 0.3%, 0.4%, 0.5%, 0.6%, 0.7%, 0.8%, 0.9%, 1.0%, 1.1%, 1.2%, 1.3%, 1.4%, 1.5% sugar. In some embodiments, the cotton fibers can comprise at most, or comprise at most about, by dry weight, 0.1%, 0.2%, 0.3%, 0.4%, 0.5%, 0.6%, 0.7%, 0.8%, 0.9%, 1.0%, 1.1%, 1.2%, 1.3%, 1.4%, 1.5% sugar. In some embodiments, the cotton fibers can comprise, by dry weight, from 0.1% to 1.1% sugar.

In some embodiments, the cotton fibers of the cotton (or engineered cotton) as described herein can comprise a threshold amount of organic acid by dry weight. In some embodiments, the cotton fibers can comprise, or comprise about, by dry weight, 0.1%, 0.2%, 0.3%, 0.4%, 0.5%, 0.6%, 0.7%, 0.8%, 0.9%, 1.0%, 1.1%, 1.2%, 1.3%, 1.4%, or 1.5% organic acid, or a range between any two foregoing values. In some embodiments, the cotton fibers can comprise at least, or comprise at least about, by dry weight, 0.1%, 0.2%, 0.3%, 0.4%, 0.5%, 0.6%, 0.7%, 0.8%, 0.9%, 1.0%, 1.1%, 1.2%, 1.3%, 1.4%, or 1.5% organic acid. In some embodiments, the cotton fibers can comprise at most, or comprise at most about, by dry weight, 0.1%, 0.2%, 0.3%, 0.4%, 0.5%, 0.6%, 0.7%, 0.8%, 0.9%, 1.0%, 1.1%, 1.2%, 1.3%, 1.4%, or 1.5% organic acid. In some embodiments, the cotton fibers can comprise, by dry weight, from 0.5% to 1.0% organic acid.

In some embodiments, the cotton fibers of the cotton (or engineered cotton) as described herein can comprise, by dry weight, 88% to 96% cellulose, 1.1% to 1.9% protein, and 0.7% to 1.2% pectic substance. In some embodiments, the cotton fibers can comprise by dry weight, 0.7% to 1.6% ash, 0.4% to 1.0% wax, 0.1% to 1.0% sugar, and 0.5% to 1.0% organic acid.

In some embodiments, the cellulose of the cotton fibers of the cotton (or engineered cotton) as described herein can comprise a threshold amount of crystalline cellulose by dry weight of the cellulose. In some embodiments, an amount of the crystalline cellulose in the cellulose can be measured by X-ray diffraction. In some embodiments, the cellulose of the cotton fibers can comprise, by dry weight, at least 65% crystalline cellulose, at least 70% crystalline cellulose, at least 75% crystalline cellulose, at least 80% crystalline cellulose, at least 85% crystalline cellulose, at least 90% crystalline cellulose, or at least 95% crystalline cellulose. In some embodiments, the cellulose of the cotton fibers can comprise at least 80% by dry weight crystalline cellulose as measured by X-ray diffraction.

In some embodiments, the cotton fibers of the cotton (or engineered cotton) as described herein can have an average strength. The average strength of the cotton fibers can be measured by a Pressley test. In some embodiments, the average strength of the cotton fibers can be measured by a zero gauge Pressley test. In some embodiments, the average strength of the cotton fibers can be measured by a ⅛-inch gauge Pressley test. A Pressley test can be performed using a Pressley tester. The Pressley tester can be a balance type tester. (The Pressley tester can comprise a beam having side A and side B, pivoted at point O. A cotton fiber can be connected at one end to side B and at another end to a clamp. The beam can be positioned initially slightly inclined, such that side B can be slightly higher than side A. A heavy rolling weight can roll down the beam toward side A, moving side B upwards. As side B rises, the clamp can move upwards. The position of the weight relative to the pivot point O and the length of side A at the point that the cotton fiber breaks can be used to calculate the strength of the cotton fiber.) The average strength of the cotton fibers can be measured by a high volume instrument (HVI) test. In some embodiments, the average strength of the cotton fibers can be measured by a ⅛-inch gauge HVI test.

In some embodiments, the cotton fibers described herein can have an average strength of, or of about, 50, 60, 65, 70, 75, 80, 85, 90, 95, or 100 Mega pounds per square inch (Mpsi), or a range between any two foregoing values. In some embodiments, the cotton fibers can have an average strength of at least 50 Mega pounds per square inch (Mpsi), at least 60 Mpsi, at least 70 Mpsi, at least 80 Mpsi, at least 90 Mpsi, or at least 100 Mpsi. In some embodiments, the cotton fibers can have an average strength of at most 50 Mega pounds per square inch (Mpsi), at most 60 Mpsi, at most 70 Mpsi, at most 80 Mpsi, at most 90 Mpsi, or at most 100 Mpsi. In some embodiments, the cotton fibers described herein can have an average strength of, or of about, 10, 11, 12, 13, 14, 15, 16, 17, 18, 19, 20 grams per tex (g/tex), or a range between any two foregoing values. In some embodiments, the cotton fibers described herein can have an average strength of at least 10 grams per tex (g/tex), at least 11 g/tex, at least 12 g/tex, at least 13 g/tex, at least 14 g/tex, at least 15 g/tex, at least 16 g/tex, at least 17 g/tex, at least 18 g/tex, at least 19 g/tex, or at least 20 g/tex. In some embodiments, the cotton fibers described herein can have an average strength of at most 10 grams per tex (g/tex), at most 11 g/tex, at most 12 g/tex, at most 13 g/tex, at most 14 g/tex, at most 15 g/tex, at most 16 g/tex, at most 17 g/tex, at most 18 g/tex, at most 19 g/tex, or at most 20 g/tex. In some embodiments, the cotton fibers can have an average strength of at least 70 Mega pounds per square inch (Mpsi). In some embodiments, the cotton fibers can have an average strength of at least 70 Mega pounds per square inch (Mpsi) as measured by a zero gauge Pressley test. In some embodiments, the cotton fibers can have an average strength of at least 15 grams per tex (g/tex). In some embodiments, the cotton fibers can have an average strength of at least 15 grams per tex (g/tex) as measured by a ⅛-inch gauge Pressley test. In some embodiments, the cotton fibers can have an average strength of at least 15 grams per tex (g/tex) as measured by a ⅛-inch gauge HVI test.

### EXAMPLES

### Example 1: Preparation of a Plant Cell Composition

From a select plant (e.g., cotton), cells are isolated by placing sterilized explants from apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and cambial meristematic cells (CMC) on a callus induction medium (e.g., a semi-solid basal salts medium) for induction. The dedifferentiated masses formed are conditioned by passing three up to five subculturing at intervals of 21-26 days on a callus growth medium (e.g., a semi-solid basal salts medium) for growth.

After cell culture stabilization, cells from a soft or friable callus are transferred into a liquid medium to form a suspension cell. Suspensions are subcultured at intervals of 15-20 days for homogenization to provide fine cell suspension culture, by filtering, pipetting/decantation, or by addition of a low concentration of pectinase. The homogeneous nature of cells in these cultures give rise to reproducible and reliable results.

### Example 2: CryoDreservation of Suspension-Cultured Cells

Cryopreservation techniques remove the need for frequent culturing and, thus, reduce the chance of microbial contamination. The protocol provided below allows the cry opreservation of over 100 cell lines simultaneously in a single day.

Suspension-cultured cells from Gossypium spp. and other species in exponentially growing phase are transferred to 15 ml tubes and centrifuged at 100×g for 1 min. Cell suspensions are handled using micropipettes with large orifice tips. The supernatant is removed, and cells are then suspended in cryoprotectant solution (LS: 2M glycerol, 0.4M sucrose) supplemented with up to 100 mM L-proline at the cell density of 10% (v/v), and incubated at room temperature for 0-120 minutes with and without shaking at 60 rpm. Aliquots (0.5 ml) of cell suspensions are dispensed into cryovials (Fisher Scientific). Cryovials containing cell suspension in LS are cooled to −35° C. at a rate of −0.5, −1, or −2° C. min using a programmable freezer. After reaching −35° C., cells are kept at −35° C. for 0, 30, or 60 minutes, and then plunged into liquid nitrogen.

In vitro dedifferentiated plant cell suspension cultures are more convenient for large-scale production, as they offer the advantage of a simplified model system for the study of plants. Cell suspension cultures contain a relatively homogeneous cell population, allowing rapid and uniform access to nutrition, precursors, growth hormones, and signal compounds for the cells.

### Example 3: Cell Recovery

The vials containing cryopreserved cells are transferred from the liquid nitrogen storage vessel into a Dewar flask containing liquid nitrogen. Each vial is transferred (one by one) to a clean 35-40° C. water bath and gently flipped several times until thawed (the last piece of ice disappears). Immediately, each vial is placed on ice again. Each vial is centrifuged at 100 g, at 4° C. for 1-2 min. The outside of each vial is wiped with 70% (vol/vol) ethanol and the supernatant from each vial is removed using a sterile Pasteur pipette. A sterile 3.5-ml transfer pipette is used to transfer two-thirds' volume of the cells by spreading or placing them as a few clusters onto the filter paper. The dish is closed and sealed with Parafilm.

The dish(es) are covered with one or two sheets of filter paper to reduce the light intensity then placed in the culture room in regular conditions (24-26° C.). After 2 days of recovery, a spatula (width of 4 mm) is used to collect some cell mass (about 100-200 mg FW) from the plate and place into a microtube for viability testing. The remaining cells are transferred with the upper filter paper to a fresh recovery dish containing recovery medium. The dishes are closed and sealed, covered with filter paper, and then returned to the culture room.

Depending on their growth rates, cells are allowed to grow for an additional number of days in the same culture room, in regular conditions (24-26° C.). When most of the filter paper is covered with a thick layer of cells, the cell mass is transferred to a fresh dish containing recovery medium without filter paper for a further 1-2 weeks under standard conditions (at this recovery stage, agarose may be replaced by agar or another gelling agent). After a recovery period of 3-9 weeks, cells are transferred to a liquid medium to initiate suspension culture.

### Example 4: Bioreactor Inoculation

For inoculum, the medium is prepared with deionized (DI) water to make a total volume of 200 mL (I L flask) and sterilized through autoclaving at 121° C. for 15 minutes. After cooling to room temperature, plant growth regulators and amino acids are added using a 0.2 pm pore size membrane filter. Twenty grams of cells are inoculated and maintained in a shaker in dark at a temperature from about 30° C. to about 35° C. at 80 rpm and left for inoculum growth. After 16 days (7 days of LAG phase and 9 days of exponential phase), the suspension is sufficiently dense for feeding the bioreactors (Titer=100 g L⁻¹, comparable a thick applesauce with no visible free medium).

An illustrative schematic of the bioreactor can be found in FIG. 2. The bioreactor is fed with in vitro cells, with sterilized medium, and air compression. The bioreactors are connected to the controller prior to inoculation, to stabilize pH 5.8 (±0.2) and to control and calibrate the flow of O2. As illustrated in the flowchart in FIG. 2, the first vessel of the inoculum train occurs at a temperature from about 30° C. to about 35° C. with a 100 g L of cells at an exponential phase. In parallel, the sterilization of the culture medium occurs at approximately 125 to approximately 140° C. and returns (stream 16) to the heat exchanger (stream 13) to cooling the medium at a temperature from about 30° C. to about 35° C. (E-103). With this, the sterile medium is ready to feed the reactors of the multiplication area (reactors R-101 to R-104).

The air for cell oxygenation is also adjusted to the process temperature in the heat exchanger (E-105) and thus is split into four different streams (streams 27, 28, 29 and 31) that feed the inoculum train (reactors R-101 to R-104).

The multiplication occurs in a duration from 5 to 12 days for cells, and the duplication time is approximately 1 day to 3 days (depending on linage(s)). These times conclude when the cell amount increases, for example, 64 times. In the end, the content is loaded to the next reactor (R-102) and so on. The last reactor (R-104) has an adjacent lung tank, where after the reaction the contents are discharged in the batch feeding tank (Tq-101) with continuous output (stream 5). Thus, during the multiplication time of the R-104 reactor, the Tq-101 is continuously unloading the cells for the next stage, the separation, at a continuous flow rate.

Table B, below, provides experimental results showing the success of inoculating a bioreactor using cotton cells in accordance with the methods disclosed herein. Table B provides details regarding the cotton varieties from which the cells were obtained, the cell growth medium which was inoculated in the bioreactor, and other relevant conditions.

TABLE B

| Variety | | | | Growth | Preliminary |
|---|---|---|---|---|---|
| Name | Quantity | Volume | Testing medium | Conditions | Growth Result |
| PD 2164 | 40 mg | 8 ml in a | 1 × MS, 1 × B5, | 30° C., dark, | poor |
| | | 25 ml bottle | 30 g/L glucose, | 180 RPM | |
| PD 2164 | 37 mg | 30 ml in a | 0.548 mg/L | 30° C., dark, | poor |
| | | 125 ml | Kinetin, 0.1 mg/L | 180 RPM | |
| | | flask | 2,4-D | | |
| Acala | 53 mg | 8 ml | 1 × MS, 1 × B5, | 30° C., dark, | poor |
| MAXXA | | | 30 g/L glucose, | 180 RPM | |
| | | | 0.548 mg/L | | |
| Acala | 31 mg | 30 ml | Kinetin, 0.1 mg/L | 30° C., dark, | poor |
| MAXXA | | | 2,4-D | 180 RPM | |
| FJA | 48 mg | 8 ml | 1 × MS, 1 × B5, | 30° C., dark, | poor |
| | | | 30 g/L glucose, | 180 RPM | |
| FJA | 41 mg | 30 ml | 0.548 mg/L | 30° C., dark, | poor |
| | | | Kinetin, 0.1 mg/L | 180 RPM | |
| | | | 2,4-D | | |
| Pima S-7 | 34 mg | 8 ml | 1 × MS, 1 × B5, | 30° C., dark, | poor |
| | | | 30 g/L glucose, | 180 RPM | |
| Pima S-7 | 48 mg | 30 ml | 0.548 mg/L | 30° C., dark, | poor |
| | | | Kinetin, 0.1 mg/L | 180 RPM | |
| | | | 2,4-D | | |
| Pima S-7 | 91 mg | 30 ml | 1 × MS, 1 × B5, | 30° C., dark, | poor |
| | | | 30 g/L glucose, | 180 RPM | |
| | | | 0.548 mg/L | | |
| | | | Kinetin | | |
| Pima S-7 | 110 mg | 30 ml | 1 × MS, 1 × B5, | 30° C., dark, | good |
| | | | 30 g/L glucose, | 180 RPM | |
| | | | 0.1 mg/L 2,4-D | | |
| Pima S-7 | 82 mg | 30 ml | 1 × MS, 1 × B5, | 30° C., light, | poor |
| | | | 30 g/L glucose, | 180 RPM | |
| | | | 0.548 mg/L | | |
| | | | Kinetin, 0.1 mg/L | | |
| | | | 2,4-D | | |
| Pima S-7 | | 8 ml in a | 1 × MS, 1 × B5, | 30° C., dark, | good |
| | | 50 ml flask | 30 g/L Sucrose, | 180 RPM | |
| | | | 0.1 mg/L Kinetin, | | |
| | | | 1 mg/L 2,4-D, | | |
| | | | 100 ml/L coconut | | |
| | | | water (B105) | | |
| Pima S-7 | | 8 ml in a | 1 × MS, 1 × B5, | 30° C., dark, | good |
| | | 50 ml flask | 20 g/L Sucrose, | 180 RPM | |
| | | | 0.1 mg/L Kinetin, | | |
| | | | 1 mg/L 2,4-D, | | |
| | | | 100 ml/L coconut | | |
| | | | water (B50) | | |
| Pima S-7 | | 8 ml in a | 1 × MS, 1 × MS, | 30° C., dark, | good |
| | | 50 ml flask | 30 g/L Sucrose, | 180 RPM | |
| | | | 0.18 mg/L NAA, | | |
| | | | 0.2 mg/L 2,4-D | | |
| | | | (M42) | | |

As shown in Table B, inoculating the bioreactor is far more efficient when done under dark conditions as opposed to light. Accordingly, the present disclosure provides methods of inoculating a bioreactor using a cotton cell culture, wherein the inoculation occurs under dark conditions.

As shown in Table B, the composition of the growth medium used when inoculating a bioreactor has an impact on cell growth. Thus, the present disclosure provides methods of inoculating a bioreactor with cotton cells, wherein the growth medium comprises plant hormones or growth regulators. As shown in Table B, when the growth medium included 2,4-dichlorophenoxyacetic acid (2,4-D), growth improved. Thus, the present disclosure provides methods of inoculating a bioreactor with cotton cells, wherein the growth medium comprises 2,4-dichlorophenoxyacetic acid (2,4-D).

As shown in Table B, the methods of the present disclosure allowed successful cell growth when inoculating a bioreactor with cells from all cotton varieties tested. Thus, the present disclosure provides methods of inoculating a bioreactor with any of the cotton varieties disclosed herein. In certain embodiments, the cotton cells used to inoculate a bioreactor in accordance with the methods disclosed herein are derived and/or obtained, in whole or in part, from at least one cotton plant of a variety selected from PAYMASTER HS26, PD 2164, SA 2413, SEALAND #1 (G.B. X G.H.), SOUTHLAND M1, STATION MILLER, TASHKENT 1, TIDEWATER 29 (G.B. X G.H.), TOOLE, WESTERN STORMPROOF, ACALA 5, ALLEN 33, CD3HCABCUH-1-89, DELTAPINE 14, DES 24, DES 56, DIXIE KING, FJA, M.U.8B UA 7-44, NC 88-95, PAYMASTER HS200, Pima S-7, Acala MAXXA, Coasland 320, or a progeny of any thereof. In certain embodiments, the cotton cells used to inoculate a bioreactor in accordance with the methods disclosed herein are derived and/or obtained, in whole or in part, from at least one cotton plant of a variety selected from PD 2164, Acala MAXXA, FJA, Pima S-7, or a progeny of any thereof.

As shown in Table B, surprisingly, cells from cotton variety Pima S-7 provided good growth when inoculating a bioreactor. This included when using milligram quantities of cotton cells to form an inoculum and across a range of growth mediums. Unexpectedly, Pima S-7 provided superior growth/inoculation compared to Acala MAXXA and FJA. Moreover, this superior growth occurred even when using the same growth medium. For example, as shown in Table B, Pima S-7 provided good growth, while Acala MAXXA and FJA showed poor growth when all were cultured using a growth medium with the same concentrations MS, B5, glucose, Kinetin, and 2,4-D. Accordingly, the present disclosure provides inoculating a bioreactor with cells derived and/or obtained, in whole or in part, from a cotton plant of the Pima S-7 variety, or a progeny thereof. Example 5: Elongation of cells

For elongation, plant cells are separated from the medium using a decanter vessel (S-101) (stream 6) and the medium can be relocated for water treatment (stream 45), as illustrated in the flowchart in FIG. 2. The elongation growth medium is added to the reactors to sterilization by autoclaving at same conditions used in multiplication step and cooling at a temperature from about 30° C. to about 35° C. for cell differentiation.

Thus, the cells from the multiplication (stream 6) feed three elongation reactors (R-105, R-106, and R-107) are represented by the reactor block (R-105) in the flowchart in FIG. 2. Each reactor receives a third of the cells and the reaction volume comprises the cells (stream 6), medium (stream 38), and air (stream 32) flows.

### Example 6: Separation and Isolation of Elongated Cells

After elongation according to Example 5, 3 tanks (Tq-102, Tq-103, and Tq-104) are fed, which in the flowchart in FIG. 2 are represented only by block Tq-102. Each tank, with volume slightly larger than those of the reactors, receives the substantially same volume of the three reactors. The output of the elongation tanks (stream 7) is routed to the second decanter (5-102). The bottom product (stream 8), comprising elongated and unelongated cells, is routed to the sieve (5-103), while the medium (stream 46) is removed to the effluent treatment. The function of the sieve is to remove unelongated and smaller cells that are not pre-fibers. The sieve (5-103) retains the elongated cells (pre-fibers) and releases all nonelongated cells (which will not become cotton fibers).

### Example 7: Maturation and Drying of Cells

In the maturation stage, as well as in the multiplication and elongation stages, a sterilized medium is used. Maturation is recognized by secondary cell wall deposition. Sugars are combined to produce cellulose, which is the main component of cotton fiber (natural glucose polymerization) that occurs inside the cell forming the secondary wall. In this process, the density of pre-fiber increases from 1.05 to 1.55 g/ml, which is the density of cotton fiber.

After maturation time, the R-108 output is directed to the buffer tank Tq-105 (FIG. 2) to enable a continuous downstream process. In the sequence, the mid-fiber mixture (stream 10) is routed to the third decanter (S-104), where the cotton fibers (stream 11) are separated from the medium (stream 48). At this stage, the fibers produced have moisture content above acceptable level (10 to 20% in water mass). To reduce the moisture content, a drying process working with air is implemented. This air passes through the cotton fibers and part of the water is removed until a moisture content of at most 5% is reached.

### Example 8: Recycling

In some embodiments, a composition created via a method described herein can be recycled. For example, in such a case, after completion of a method or step of a method, an aliquot of a composition is reserved and re-introduced into an earlier step in a method. In some cases, an aliquot of cells unsuccessful in induction, growth, elongation, or maturation is reserved and re-introduced into an earlier step in a method.

### Example 9: Production of Cotton Fiber from Cotton Ovule Cells

Tables C, D, and E, below, show the results of growth and elongation of cotton cell cultures in accordance with the methods disclosed herein. For each growth result, cotton ovule cells, which may include ovule epidermal cells, were mechanically extracted from a cotton boll. The extracted cotton ovule cells were cultured, multiplied, and in some cases, elongated. Each table provides the genotype/cultivar and variety name from which the cotton cell cultures were originally obtained. The tables also provide the ovule location from which the cells were taken from a parental cotton plant for the cell cultures.

TABLE C
Cotton cell culture results from cells
cultured from an upper ovule location.

| Variety Name | Ovule location | Growth Results |
|---|---|---|
| PAYMASTER HS26 | Upper | some |
| PD 2164 | Upper | excellent |
| SA 2413 | Upper | poor |
| SEALAND #1 (G.B. × G.H.) | Upper | some/fiber |
| SOUTHLAND M1 | Upper | good |
| STATION MILLER | Upper | some |
| TASHKENT 1 | Upper | some |
| TIDEWATER 29 (G.B. × | Upper | some |
| G.H.) | | |
| TOOLE | Upper | poor |
| WESTERN STORMPROOF | Upper | some |
| ACALA 5 | Upper | excellent/fiber |
| ALLEN 33 | Upper | some |
| CD3HCABCUH-1-89 | Upper | good |
| DELTAPINE 14 | Upper | some |
| DES 24 | Upper | some |
| DES 56 | Upper | poor |
| DIXIE KING | Upper | some |
| FJA | Upper | some |
| M.U.8B UA 7-44 | Upper | some |
| NC 88-95 | Upper | some |
| PAYMASTER HS200 | Upper | some |
| Pima S-7 | Upper | some |
| Acala MAXXA | Upper | some |
| Coasland 320 | Upper | poor |

TABLE D
Cotton cell culture results from cells
cultured from a middle ovule location.

| Variety Name | Ovule location | Growth |
|---|---|---|
| PAYMASTER HS26 | Middle | some |
| PD 2164 | Middle | excellent |
| SA 2413 | Middle | some/fiber |
| SEALAND #1 (G.B. × G.H.) | Middle | some/fiber |
| SOUTHLAND M1 | Middle | good |
| STATION MILLER | Middle | some |
| TASHKENT 1 | Middle | some |
| TIDEWATER 29 (G.B. × | Middle | some |
| G.H.) | | |
| TOOLE | Middle | some/fiber |
| WESTERN STORMPROOF | Middle | some |
| ACALA 5 | Middle | some |
| ALLEN 33 | Middle | some |
| CD3HCABCUH-1-89 | Middle | good |
| DELTAPINE 14 | Middle | some |
| DES 24 | Middle | some |
| DES 56 | Middle | poor |
| DIXIE KING | Middle | some |
| FJA | Middle | excellent |
| M.U.8B UA 7-44 | Middle | some/fiber |
| NC 88-95 | Middle | some |
| PAYMASTER HS200 | Middle | some |
| Pima S-7 | Middle | some |
| Acala MAXXA | Middle | some |
| Coasland 320 | Middle | poor |

TABLE E
Cotton cell culture results from cells
cultured from a bottom ovule location.

| Variety Name | Ovule location | Growth |
|---|---|---|
| PAYMASTER HS26 | Bottom | some |
| PD 2164 | Bottom | excellent |
| SA 2413 | Bottom | some |
| SEALAND #1 (G.B. × G.H.) | Bottom | poor |
| SOUTHLAND M1 | Bottom | excellent |
| STATION MILLER | Bottom | some |
| TASHKENT 1 | Bottom | good |
| TIDEWATER 29 (G.B. × | Bottom | some |
| G.H.) | | |
| TOOLE | Bottom | some/fiber |
| WESTERN STORMPROOF | Bottom | good |
| ACALA 5 | Bottom | excellent/fiber |
| ALLEN 33 | Bottom | some |
| CD3HCABCUH-1-89 | Bottom | excellent |
| DELTAPINE 14 | Bottom | some |
| DES 24 | Bottom | some |
| DES 56 | Bottom | poor |
| DIXIE KING | Bottom | some/fiber |
| FJA | Bottom | excellent |
| M.U.8B UA 7-44 | Bottom | some/fiber |
| NC 88-95 | Bottom | some |
| PAYMASTER HS200 | Bottom | good |
| Pima S-7 | Bottom | excellent |
| Acala MAXXA | Bottom | excellent |
| Coasland 320 | Bottom | poor |

As shown in Tables C, D, E, all varieties were successfully grown in accordance with the methods of the present disclosure. Thus, the present disclosure provides cotton, methods of growing cotton in accordance with any of the method/protocols provided herein, and persistent cell lines, wherein the cotton cells are derived and/or obtained, in whole or in part, which can be used across a range of cotton species and varietals. Accordingly, the in vitro methods of cotton production can use cotton cells derived from a cotton plant of any varietal, including one selected from PAYMASTER HS26, PD 2164, SA 2413, SEALAND #1 (G.B. X G.H), SOUTHLAND M1, STATION MILLER, TASHKENT 1, TIDEWATER 29 (G.B. X G.H), TOOLE, WESTERN STORMPROOF, ACALA 5, ALLEN 33, CD3HCABCUH-1-89, DELTAPINE 14, DES 24, DES 56, DIXIE KING, FJA, M.U.8B UA 7-44, NC 88-95, PAYMASTER HS200, Pima S-7, Acala MAXXA, Coasland 320, and or a progeny of any thereof.

As shown in Tables C, D, and E, certain varieties produced good or excellent growth. Thus, in certain embodiments, the cotton cells are derived and/or obtained, in whole or in part, from at least one cotton plant of a variety selected from PD 2164, SOUTHLAND M1, ACALA 5, CD3HCABCUH-1-89, FJA, TASHKENT 1, WESTERN STORMPROOF, PAYMASTER HS200, Pima S-7, and Acala MAXXA, or a progeny of any thereof. Certain varieties produced excellent growth. Thus, in certain embodiments, the cotton cells are derived and/or obtained, in whole or in part, from at least one cotton plant of a variety selected from PD 2164, ACALA 5, SOUTHLAND M1, CD3HCABCUH-1-89, FJA, Pima S-7, and Acala MAXXA, or a progeny of any thereof.

As shown in Table C, certain varieties produced good or excellent growth using cells obtained from an ovule, which may include ovule epidermal cells, located on the upper/top third of a boll, e.g., distal from the location on the boll to which it connects or connected to the stem of a cotton plant. Thus, in certain embodiments, the cotton cells are derived and/or obtained, in whole or in part, from ovule cells and/or ovule epidermal cells obtained from the top third of a cotton boll from at least one cotton plant of a variety selected from PD 2164, SOUTHLAND M1, ACALA 5, and CD3HCABCUH-1-89, or a progeny of any thereof. As shown in Table C, certain varieties produced excellent growth using ovule cells and/or ovule epidermal cells obtained from the top third of a cotton boll. Thus, in certain embodiments, the cotton cells are derived and/or obtained, in whole or in part, from ovule and/or ovule epidermal cells obtained from the top third of a cotton boll from at least on cotton plant of a variety selected from PD 2164 and ACALA 5, or a progeny of any thereof.

As shown in Table D, certain varieties produced good or excellent growth using ovule cells and/or ovule epidermal cells obtained from the middle third of the cotton boll. Thus, in certain embodiments, the cotton cells are derived and/or obtained, in whole or in part, from ovule cells and/or ovule epidermal cells obtained from the middle third of a cotton boll from a cotton plant of a variety selected from at least one cotton plant of a variety selected from PD 2164, SOUTHLAND M1, CD3HCABCUH-1-89, FJA, or a progeny of any thereof. As shown in Table D, certain varieties produced excellent growth using ovule cells and/or ovule epidermal cells obtained from the middle third of a cotton boll. Thus, in certain embodiments, the cotton cells are derived and/or obtained, in whole or in part, from ovule cells and/or ovule epidermal cells obtained from the middle third of a cotton boll from at least on cotton plant of a variety selected from PD 2164 and FJA, or a progeny of any thereof.

As shown in Table E, certain varieties produced good or excellent growth using ovule cells and/or ovule epidermal cells obtained from the bottom third of the boll. Thus, in certain embodiments, the cotton cells are derived and/or obtained, in whole or in part, from ovule cells and/or ovule epidermal cells obtained from the bottom third of a cotton boll from a cotton plant of a variety selected from at least one cotton plant of a variety selected from PD 2164, SOUTHLAND M1, TASHKENT 1, WESTERN STORMPROOF, AC ALA 5, CD3HCABCUH-1-89, FJA, Pima S-7, Acala MAXXA, or a progeny of any thereof. As shown in Table E, certain varieties produced excellent growth using ovule cells and/or ovule epidermal cells obtained from the bottom third of a cotton boll. Thus, in certain embodiments, the cotton cells are derived and/or obtained, in whole or in part, from ovule cells and/or ovule epidermal cells obtained from the bottom third of a cotton boll from at least on cotton plant of a variety selected from PD 2164, SOUTHLAND M1, ACALA 5, CD3HCABCUH-1-89, FJA, Pima S-7, and Acala MAXXA, or a progeny of any thereof.

As shown in Tables C, D, and E, certain varieties produced good or excellent growth using cells from more than one ovule location. Thus, in certain embodiments, the cotton cells are derived and/or obtained, in whole or in part, from ovule cells and/or ovule epidermal cells obtained from at least one cotton plant of a variety selected from PD 2164, SOUTHLAND M1, ACALA 5, FJA, or a progeny of any thereof.

As shown in Tables C, D, and E, certain varieties quickly produced detectable levels of fiber in the grown cells. Accordingly, in certain embodiments, the cotton cells are derived and/or obtained, in whole or in part, from at least one cotton plant of a variety selected from SEALAND #1 (G.B. X G.H.), ACALA 5, SA 2413, TOOLE, M.U.8B UA 7-44, DIXIE KING, or a progeny of any thereof. As shown in Tables C, D, and E, certain varieties quickly produced detectable levels of fiber in the grown cells. As shown in Tables C and E, ACALA 5 showed both quickly detectable levels of fiber and excellent growth.

Thus, as the foregoing examples reveal, the presently disclosed in vitro methods of cotton production are amenable to using cotton cells with varying traits and genetic backgrounds. Accordingly, these methods can find clear use using cotton cells that express selected genes of interest.

### Example 10: Producing Colored Cotton Fiber

The present invention includes the transformation and selection of transformed cells, and induction of those cells to elongate to form fiber.

In an exemplary assay cotton cells are obtained from the cotton varietals listed in Table F in accordance with the methods described herein. However, cotton cells obtained from any cotton varietal, including those specifically listed herein (such as those listed in Tables B-E), can be used in accordance with the methods of the invention.

TABLE F
Cotton varietals
Variety Name

TM-1
DP-50
DP-51
Coker 315
DP-90

The obtained cells are from cotton plants of the varietals in Table F and/or are the progeny of cells obtained from plants of these varietals. The cotton cells are obtained or are the progeny of cotton cells obtained from cotton plant or explant apical meristems, cotyledons, young leaves, hypocotyls, ovules, stems, mature leaves, flower, flower stalks, floral whorls, roots, bulbs, germinated seeds, somatic and zygotic embryo, and/or cambial meristematic cells (CMC). The obtained cells are isolated and proliferated with or without a callus phase.

The cells derived from suspension cultures or calli are then subject to a transformation to introduce a plurality of expression constructs into the cotton cells The transformation method used is either an Agrobacterium or biolistic transformation method based on prior published studies of cotton transformation to regenerate whole plants, however, in this case, cotton cells are transformed and elongated to form fiber rather than whole plants (Jin, 2005; Leelavathi, 2004; Finer, 1990).

In methods using d zYz/zcVc vz/zzz-mediated transformation, the cells are co-cultivated with an Agrobacterium carrying vector that includes three expression constructs. One of the constructs produces a red chromoprotein/pigment, one a green chromoprotein/pigment, and one a blue chromoprotein/pigment. Each construct is independently inducible and tunable. In certain aspects, each expression construct is operably connected to an inducible promoter. In certain aspects, the promoters include, for example, promoters that show preferential tissue and/or temporal expression in cotton plants or cotton cells. In methods using biolistic transformation, the cells are subject to bombardment with the genes of interest and the selection gene(s).

After transformation, the cells are grown with media containing hormones to induce cell growth, a selection agent to inhibit the growth of untransformed cells due to the selection gene(s). For methods that employ d z′o/zzzc/cz′zz/zzz-mediated transformation, the media also includes antibiotic(s) to inhibit the growth of excess Agrobacterium.

After incorporation of the expression constructs, a desired color for the cotton fiber produced from the cells is chosen. The cotton cells are contacted with varying concentrations of Inducers A, B, and C. Each different inducer induces a separate gene construct, which leads to the production of a particular pigment/chrom oprotein in the cotton fiber with saturation that corresponds to the concentration of inducer used for the construct. By adjusting the various concentrations of inducers, and the resulting pigments/chromoproteins generated in the cotton fiber, cotton fiber of any color can be created. The concentrations of each inducer required to produce cotton fiber of a particular color are stored in a database, such that cotton fiber of a particular color can quickly be replicated using the disclosed in vitro methods of colored cotton production.

The cells are cultured, the constructs induced, and the cells caused to elongate in order to produce cotton fiber of a desired color.

### Example 11: Low Trash Cotton

Four separate harvests of cotton fiber were obtained from separate batches of cotton cells produced in a bioreactor using the methods as set forth in Examples 1-9. Samples of the cotton fiber were obtained from each harvest and assessed using an Advanced Fiber Information System (AFIS). AFIS is an industry-standard instrument system for analyzing various fiber quality parameters of harvested cotton. Among the fiber qualities AFIS is able to measure is the trash content of cotton fiber.

Hand-picked cotton from a field was also analyzed by AFIS and used as a control. The AFIS-detected trash content (greater than 500 microns) for each sample are provided in Table G.

TABLE G

| Sample | Harvest 1 | Harvest 2 | Harvest 3 | Harvest 4 | Control |
|---|---|---|---|---|---|
| Trash | 82 | 153 | 255 | 236 | 251 |
| content | | | | | |
| (Cnt/g) | | | | | |

As shown, surprisingly, the present inventors discovered that the methods of the invention produce cotton fiber, with less than ⅓ the trash content of even hand-picked cotton produced using in planta methods. Example 12: Seed coat neps

Five separate harvests of cotton fiber were obtained from separate batches of cotton cells produced in a bioreactor using the methods as set forth in Examples 1-9. Samples of the cotton fiber were obtained from each harvest and assessed using an Advanced Fiber Information System (AFIS). Among the fiber qualities AFIS is able to measure is number of seed coat neps in cotton fiber.

Hand-picked cotton from a field was also analyzed by AFIS and used as a control. The AFIS-detected seed coat nep counts for each sample are provided in Table H.

TABLE H

| | Har- | Har- | Har- | Har- | Bleached | |
|---|---|---|---|---|---|---|
| Sample | vest 1 | vest 2 | vest 3 | vest 4 | Harvest | Control |
| Seed Coat Neps | 90 | 38 | 27 | 34 | 16 | 34 |
| (Cnt/g) | | | | | | |

As shown, surprisingly, the present inventors discovered that the methods of the invention produced cotton fiber with a comparative seed coat nep content relative to hand-picked cotton produced using in planta methods. However, surprisingly, a light bleach treatment was able to halve the amount of seed coat neps.

Furthermore, the present inventors discovered that, if cotton ovules are identified and removed from the culture before harvest, the number of seed coat neps likewise greatly diminished in the harvested cotton.

### Example 13: Other Fiber Qualities

Four separate harvests of cotton fiber were obtained from separate batches of cotton cells produced in a bioreactor using the methods as set forth in Examples 1-9. Samples of the cotton fiber were obtained from each harvest and assessed using an Advanced Fiber Information System (AFIS). Among the fiber qualities AFIS is able to measure is the short fiber content, maturity, fineness, and immature fiber content.

Hand-picked cotton from a field was also analyzed by AFIS and used as a control. The AFIS-detected short fiber content, maturity, fineness, and immature fiber content for each sample are provided in Table I.

TABLE I

| Sample | Harvest 1 | Harvest 2 | Harvest 3 | Harvest 4 | Control |
|---|---|---|---|---|---|
| Short Fiber | 67 | 17 | 10.9 | 17.2 | 8.8 |
| Content (%) | | | | | |
| Fineness | 139 | 124 | 129 | 123 | 157 |
| (Mtex) | | | | | |
| Immature Fiber | 12.8 | 10.1 | 8.5 | 10.2 | 4.8 |
| Content (%) | | | | | |
| Maturity | 0.68 | 0.75 | 0.74 | 0.7 | 0.88 |

As shown, surprisingly, the present inventors discovered that the methods of the invention produced cotton fiber with a comparative short fiber content, fineness, and immature fiber content relative to the hand-picked, traditionally-grown cotton. These results were despite the comparative immaturity of the cotton fiber produced using the methods of the invention. Nevertheless, as the methods of the invention are entirely controlled, rather than at the whims of field conditions, cotton fiber can be harvested when a higher percentage of the cotton fibers are shown to be mature. This should improve all cotton fiber qualities. As relative immature cotton grown using methods of the invention provide comparable or improved qualities over field-grown, hand-picked cotton, they represent a cost- and environmentally-efficient means to produce high-quality cotton fiber.

Various publications are referenced throughout this application. Full citations for select references may be found listed at the end of the specification and preceding the claims. The disclosures of all referenced publication are hereby incorporated by reference in their entirety.

### REFERENCES

Bouchabke-Coussa, O., Obellianne, M., Linderme, D., Montes, E., Maia-Grondard, A., Vilaine, F., & Pannetier, C. (2013). Wuschel overexpression promotes somatic embryogenesis and induces organogenesis in cotton (Gossypium hirsutum L.) tissues cultured in vitro. Plant cell reports, 32(5), 675-686.
Cai, C., Guo, W. & Zhang, B (2018). Genome-wide identification and characterization of SPL transcription factor family and their evolution and expression profiling analysis in cotton. Sci Rep 8, 762.
Deng F, Tu L, Tan J, Li Y, Nie Y, Zhang X (2012). GbPDF1 is involved in cotton fiber initiation via the core cis-element HDZIP2ATATHB2. Plant Physiology February 2012, 158 (2) 890-904.
Finer J J & McMullen M D (1990) Transformation of cotton (Gossypium hirsutum L.) via particle bombardment. Plant Cell Rep. 8: 586-589.
Gordon-Kamm, B., Sardesai, N., Arling, M., Lowe, K., Hoerster, G., Betts, S., & Jones, A. T. (2019). Using Morphogenic Genes to Improve Recovery and Regeneration of Transgenic Plants. Plants (Basel, Switzerland), 8(2), 38.
Hoerster, G., Wang, N., Ryan, L. et al. Use of non-integrating Zm-Wus2 vectors to enhance maize transformation. In Vitro Cell. Dev. Biol. -Plant 56, 265-279 (2020).
Jin et al., Molecular and Functional Dissection of the Maize B Chromosome Centromere, The Plant Cell May 2005, 17 (5) 1412-1423.
Khalilian, A., Marshall, M., Williams, P., Greene, J. and Porter, P. (2018) Cotton Production Systems for Soil and Energy Conservation in Coastal Plain Soils. American Journal of Plant Sciences, 9, 1500-1513.
Leelavathi et al., A simple and rapid Agrobacterium-mediated transformation protocol for cotton (Gossypium hirsutum L.): embryogenic calli as a source to generate large numbers of transgenic plants. Plant Cell Rep. 2004 February; 22(7):465-70.
Liu, Z.-H., Chen, Y., Wang, N.-N., Chen, Y.-H., Wei, N., Lu, R., Li, Y. and Li, X.-B. (2020). A basic helix-loop-helix protein (GhFP1) promotes fibre elongation of cotton (Gossypium hirsutum) by modulating brassinosteroid biosynthesis and signalling. New Phytol, 225: 2439-2452.
Lowe, K., Wu, E., Wang, N., Hoerster, G., Hastings, C., Cho, M. J., Scelonge, C., Lenderts, B., Chamberlin, M., Cushatt, J., Wang, L., Ryan, L., Khan, T., Chow-Yiu, J., Hua, W., Yu, M., Banh, J., Bao, Z., Brink, K., Igo, E., . . . Gordon-Kamm, W. (2016). Morphogenic Regulators Baby boom and Wuschel Improve Monocot Transformation. The Plant cell, 28(9), 1998-2015.
Lowe, K., La Rota, M., Hoerster, G., Hastings, C., Wang, N., Chamberlin, M., Wu, E., Jones, T., & Gordon-Kamm, W. (2018). Rapid genotype “independent” Zea mays L. (maize) transformation via direct somatic embryogenesis. In vitro cellular & developmental biology. Plant: journal of the Tissue Culture Association, 54(3), 240-252.
Luo, K., Zhang, G., Deng, W. et al. (2008). Functional characterization of a cotton late embryogenesis-abundant D I 13 gene promoter in transgenic tobacco. Plant Cell Rep 27, 707-717.
Pu, L., Li, Q., Fan, X., Yang, W., Xue, Y. (2008). The R2R3 MYB transcription factor GhMYB109 is required for cotton fiber development. Genetics 180(2): 811-820 (2008).
Shan, C., Shangguan, X., Zhao, B. et al. (2014). Control of cotton fibre elongation by a homeodomain transcription factor GhHOX3. Nat Commun 5, 5519.
Walford, S. A., Wu, Y., Llewellyn, D. J., & Dennis, E. S. (2012). Epidermal cell differentiation in cotton mediated by the homeodomain leucine zipper gene, GhHD-1. The Plant Journal: for cell and molecular biology, 71(3), 464-478.
Wang, S., Wang, J. W., Yu, N., Li, C. H., Luo, B., Gou, J. Y., Wang, L. L, & Chen, X. Y. (2004). Control of plant trichome development by a cotton fiber MYB gene. The Plant cell, 16(9), 2323-2334.
Zhang, M., Zheng, X., Song, S., Zeng, Q., Hou, L., Li, D., Zhao, J., Wei, Y., Li, X.,
Luo, M., Xiao, Y., Luo, X., Zhang, J., Xiang, C., & Pei, Y. (2011). Spatiotemporal manipulation of auxin biosynthesis in cotton ovule epidermal cells enhances fiber yield and quality. Nature biotechnology, 29(5), 453-458.
Zeng, Y D., Sun, J L., Bu, S H. et al. EcoTILLING revealed SNPs in GhSus genes that are associated with fiber- and seed-related traits in upland cotton. Sci Rep 6, 29250 (2016).
Zheng, W., Zhang, X., Yang, Z., Wu, J., Li, F., Duan, L., Liu, C., Lu, L., Zhang, C., & Li, F. (2014). AtWuschel promotes formation of the embryogenic callus in Gossypium hirsutum. PloS one, 9(1), e87502.
Wan, P., Xu, D., Cong, S., Jiang, Y., Huang, Y., Wang, J., Wu, H., Wang, L., Wu, K., Carriere, Y., Mathias, A., Li, X., Bruce E. Tabashnik, B. (2017). Hybridizing transgenic Bt cotton with non-Bt cotton counters resistance in pink bollworm. PNAS, 114 (21) 5413-5418.

What is claimed is:

1. A composition comprising harvested cotton fiber from cotton cells elongated in a bioreactor, wherein said cotton fiber has a trash content of less than 250 Count/gram (Cnt/g) dry mass.

2. The composition of claim 1, wherein the harvested cotton fiber has a trash content of between about 25 Cnt/g dry weight and about 50 Cnt/g dry weight; 50 Cnt/g dry weight and about 75 Cnt/g dry weight; 75 Cnt/g dry weight and about 100 Cnt/g dry weight; 100 Cnt/g dry weight and about 125 Cnt/g dry weight; 125 Cnt/g dry weight and about 150 Cnt/g dry weight; 150 Cnt/g dry weight and about 175 Cnt/g dry weight; 175 Cnt/g dry weight and about 200 Cnt/g dry weight; 200 Cnt/g dry weight and about 225 Cnt/g dry weight; or 225 Cnt/g dry weight and about 250 Cnt/g dry weight.

3. The composition of claim 2, wherein prior to harvest cotton ovule cells are removed from the composition.

4. The composition of claim 2, wherein the harvested cotton has a seed coat nep content of between about 15 Cnt/g and about 90 Cnt/g.

5. The composition of claim 2, wherein the harvested cotton comprises at most 3% by dry weight of a trash content.

6. The composition of claim 5, wherein the harvested cotton comprises at most 1% by dry weight of a trash content.

7. The composition of claim 2, wherein the harvested cotton comprises at most 8.5% by dry weigh a short fiber content (SFC).

8. The composition of claim 2, wherein the harvested cotton comprises an immature fiber content of at most about 10%.

9. The composition of claim 8, wherein the harvested cotton comprises an immature fiber content of at most about 8%.

10. A method for producing cotton fiber, the method comprising: inoculating a bioreactor with cotton cells; multiplying the cells in the bioreactor; elongating the multiplied cells; and harvesting cotton fiber from the elongated cells, wherein the harvested cotton fiber has a trash content of less than 250 Count/gram (Cnt/g) dry mass.

11. The method of claim 10, wherein the harvested cotton fiber has a trash content of between about 25 Cnt/g dry weight and about 50 Cnt/g dry weight; 50 Cnt/g dry weight and about 75 Cnt/g dry weight; 75 Cnt/g dry weight and about 100 Cnt/g dry weight; 100 Cnt/g dry weight and about 125 Cnt/g dry weight; 125 Cnt/g dry weight and about 150 Cnt/g dry weight; 150 Cnt/g dry weight and about 175 Cnt/g dry weight; 175 Cnt/g dry weight and about 200 Cnt/g dry weight; 200 Cnt/g dry weight and about 225 Cnt/g dry weight; or 225 Cnt/g dry weight and about 250 Cnt/g dry weight.

12. The method of claim 11, wherein the bioreactor is inoculated with cotton cells comprising cotton ovule cells.

13. The method of claim 12, further comprising identifying and removing non-elongated cells prior to harvesting the cotton fiber, thereby removing cotton ovules from the bioreactor.

14. The method of claim 13, wherein the method produces at least 1 kilogram of cotton fiber for every 4,000 liters of water used in the method.

15. The method of claim 14, wherein the method produces at least 1 kilogram of cotton fiber for between every 2,000 and 4,000 of water used in the method.

16. The method of claim 11, wherein the bioreactor is inoculated with cells from a proliferating cell aggregate.

17. The method of claim 16, wherein the proliferating cell aggregate is a friable callus.

18. The method of claim 17, further comprising: obtaining cells from a cotton explant; and contacting the cells from the cotton explant with a callus induction medium to produce the friable callus.

19. The method of claim 18, wherein the cells from a cotton explant are from cotton apical meristems, cotyledons, young leaves, hypocotyls, ovules, stems, mature leaves, flower, flower stalks, floral whorls, roots, bulbs, germinated seeds, somatic and zygotic embryo, and/or cambial meristematic cells (CMC).

20. The method of claim 19, wherein the method further comprises: dissociating cells from the friable callus; and culturing the dissociated cells.
